(19) United States
(12) Patent Application Publication
(10) Pub. No.: US 2024/0157023 A1
(43) Pub. Date: May 16, 2024

(54) THE COMBINED INFLUENCE OF VISCOELASTIC AND ADHESIVE CUES ON FIBROBLAST SPREADING AND FOCAL ADHESION FORMATION

(71) Applicant: University of Virginia Patent Foundation, Charlottesville, VA (US)

(72) Inventors: Steven R. Caliari, Charlottesville, VA (US); Erica Hui, Charlottesville, VA (US); Thomas H. Barker, Charlottesville, VA (US)

(73) Assignee: University of Virginia Patent Foundation, Charlottesville, VA (US)

(21) Appl. No.: 18/277,752

(22) PCT Filed: Feb. 17, 2022
(86) PCT No.: PCT/US2022/016872

§ 371 Date: Aug. 17, 2023

Related U.S. Application Data
(60) Provisional application No. 63/150,315, filed on Feb. 17, 2021.

(51) Int. Cl. A61L 26/00; A61K 41/00

(52) U.S. Cl. CPC A61L 26/0061; A61K 41/00; A61L 26/0023; A61L 26/008; A61L 2300/252; A61L 2400/06

(57) ABSTRACT

Disclosed are phototunable hydrogels, compositions that include the same, and methods for using the same for treating wounds and/or injuries, for inhibiting formation of scar tissue at wound sites, for inhibiting fibrosis in subjects in need thereof, for inhibiting lung fibrosis and/or scarring in subject in need thereof, for inhibiting formation of myofibroblasts from fibroblasts, and for inhibiting expression of α-smooth muscle actin (α-SMA) and/or type I collagen in fibroblasts.

### CROSS REFERENCE TO RELATED APPLICATION

The presently disclosed subject matter claims the benefit of U.S. Provisional Patent Application Ser. No. 63/150,315, filed Feb. 17, 2021, the disclosure of which incorporated herein by reference in its entirety.

### GOVERNMENT INTEREST

This invention was made with government support under Grant No. GM138187 awarded by The National Institutes of Health. The government has certain rights in the invention.

### REFERENCE TO SEQUENCE LISTING SUBMITTED ELECTRONICALLY

The content of the electronically submitted sequence listing in ASCII text file (Name: 3062_151_PCT_ST25.txt; Size: 22 kilobytes; and Date of Creation: Feb. 17, 2022) filed with the instant application is incorporated herein by reference in its entirety.

### BACKGROUND

Tissue fibrosis is a pathological scarring process characterized by the excessive deposition of crosslinked extracellular matrix (ECM) proteins leading to progressive matrix stiffening and decreased viscoelasticity (Liu et al., 2010; Wynn & Ramalingam, 2012; Wells, 2014; Perepelyuk et al., 2016; Fiore et al., 2018; Hilster et al., 2020). These aberrant changes in tissue mechanics detrimentally impact organ function, contributing to the role fibrosis plays in nearly half of all deaths in the developed world (Wynn, 2008; Martinez et al., 2017; Henderson et al., 2020). Reciprocal interactions between fibroblasts and their surrounding extracellular microenvironment actively drive a cascade of biochemical and biophysical signaling events to direct both normal and fibrogenic behaviors including adhesion, spreading, focal adhesion organization, and activation into fibrosis-promoting myofibroblasts (Hinz & Gabbiani, 2003; Ingber, 2006; Brown et al., 2011; Duscher et al., 2014; Humphrey et al., 2014; Jansen et al., 2017). However, delineating the specific environmental regulators of fibroblast behavior is difficult in multifaceted tissue milieus.

Numerous in vitro studies have used hydrogel biomaterials to deconstruct complex in vivo cellular microenvironments to better understand the individual and combined influence of biophysical factors such as stiffness and viscoelasticity on driving fibrogenic cell behaviors (Cameron et al., 2011; Baker & Chen, 2012; Caliari & Burdick, 2016; Caliari et al., 2016b; Yeh et al., 2017; Charrier et al., 2018; Gong et al., 2018; Chaudhuri et al., 2020). It is well understood that stiffer microenvironments can guide mechanotransduction by providing biophysical cues for fibroblast activation. Culturing cells atop substrates of increasing stiffness promotes increased spreading, actin stress fiber organization, and nuclear localization of transcriptional cofactors regulating the expression of fibrogenic genes encoding α-smooth muscle actin (α-SMA) and type I collagen (Wells, 2008; Kloxin et al., 2010; Olsen et al., 2011; Balestrini et al., 2012; Wen et al., 2014; Caliari et al., 2016c; Travers et al., 2016; Yeh et al., 2017). While many studies of mechanotransduction use covalently-crosslinked hydrogels that behave as linearly elastic solids, tissues are viscoelastic, meaning they exhibit both elastic solid and viscous liquid-like behaviors such as stress relaxation (Fiore et al., 2018; Islam et al., 2020; Zhu et al., 2020). Seminal studies incorporating viscoelasticity into hydrogels showed that, compared to stiffness-matched elastic controls, cells displayed reduced spreading and expression of disease-relevant markers such as α-SMA with increasing loss modulus (viscoelasticity) due to reduced cellular contractility as a result of viscous dissipation (Charrier et al., 2018; Hui et al., 2019), highlighting the importance of viscoelasticity in disease mechanobiology.

While stiffness and viscoelasticity are well-established regulators of cell behavior, comparatively little attention has been paid to engineering hydrogels that can control cell adhesive interactions through preferential integrin engagement. Integrins are transmembrane proteins composed of α and β subunits that bind to the ECM and serve as conduits for biochemical and mechanical signaling between cells and the ECM (Rustad et al., 2013; Kechagia et al., 2019). Importantly, integrin-based adhesions enable the conversion of complex biophysical cues, such as matrix mechanics and viscoelasticity, into chemical signals through mechanotransduction (Roca-Cusachs et al., 2012; Henderson & Sheppard, 2013; Conroy et al., 2016; Sun et al., 2016; Asano et al., 2017). Integrin engagement and clustering facilitates the recruitment and formation of force-dependent focal adhesions (FAs) composed of proteins including paxillin, which play an important role in regulating cell behaviors such as spreading, contraction, migration, and differentiation (Ingber, 2006; Paszek et al., 2009; Duscher et al., 2014; Seong et al., 2013; Humphrey et al., 2014; Jansen et al., 2017). As nascent cell-matrix adhesions (<0.25 μm) mature into stable and larger FAs (1-5 μm), this strengthens integrin-FA-cytoskeletal linkages, facilitating actin polymerization and stress fiber organization, nuclear localization of transcriptional mechanoregulators, and the transcription of fibrogenic genes that ultimately results in dysregulated ECM production and organ failure (Gardel et al., 2010; Harjanto & Zaman, 2010; Roca-Cusachs et al., 2012; Humphrey et al., 2014; Kishi et al., 2016; Panciera et al., 2017; Driscoll et al., 2020). While many synthetic hydrogels are engineered to support integrin-mediated cell attachment by incorporating the fibronectin-derived RGD peptide, this may inadvertently convolute mechanobiology studies due to its inefficient cell binding affinity compared to longer peptide or protein domains as well as its ability to non-specifically bind multiple integrin heterodimers (Ruoslahti, 1996). Recent work has shown that provisional matrix proteins such as fibronectin (Fn) are upregulated during early stages of tissue remodeling and that preferential engagement of Fn-associated integrins (e.g., αvβ3 vs α5β1) caused by tension-stimulated conformational changes can influence fibrosis mechanoregulation (Roca-Cusachs et al., 2012; Conroy et al., 2016; Fiore et al., 2018; Leiphart et al., 2019). In particular, engagement of the αv integrin has been shown to promote integrin-mediated myofibroblast contractility (Paszek et al., 2009; Harjanto & Zaman, 2010; Henderson et al., 2013; Fiore et al., 2018), mechanoactivation of latent transforming growth factor-beta 1 (TGF-β1; Arora et al., 1999; Fernandez & Eickelberg, 2012; Henderson & Sheppard, 2013), and expression and organization of α-SMA stress fibers, a hallmark of myofibroblast activation (Scaffidi et al., 2001; Balestrini et al., 2012; Deng et al., 2015).

While several studies, including from our group (Hui et al., 2019), have highlighted the importance of stiffness and viscoelasticity in directing cell behavior, an approach to independently manipulate stiffness, viscoelasticity, and integrin engagement in a single system has not been developed. To address this challenge, disclosed herein is a phototunable viscoelastic hydrogel platform that can be employed to deconstruct the complexity of native tissue toward understanding the individual and combined roles of cell-instructive cues including stiffness, viscoelasticity, and integrin-binding ligand presentation. This system was also employed to determine how multiple mechanoregulatory cues work together to guide cellular behavior in the context of fibroblast activation.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to phototunable hydrogels. In some embodiments, the phototunable hydrogels comprise, consist essentially of, or consist of a norbornene-functionalized hyaluronic acid (HA) backbone and optionally one or more additional functional moieties attached thereto, wherein the one or more additional functional moieties are selected from the group consisting of β-cyclodextrin and adamantane, or any combination thereof; and one or more peptides and/or polypeptide fragments, wherein the one or more peptides and/or polypeptide fragments are selected from the group consisting of an RGD peptide and a fibronectin polypeptide fragment, optionally wherein the fibronectin polypeptide fragment is selected from the group consisting of an α5β1 peptide and an αvβ3 peptide, or any combination thereof, and further optionally wherein the fibronectin polypeptide fragment is thiolated. In some embodiments, at least two norbornene moieties are crosslinked to each other, optionally with a dithiol crosslinker. In some embodiments, the dithiol crosslinker comprises a covalent crosslink that results from light-mediated thiol-ene addition. In some embodiments, the norbornene-functionalized HA backbone lacks β-cyclodextrin and adamantane and the phototunable hydrogel is an elastic phototunable hydrogel. In some embodiments, the norbornene-functionalized HA backbone comprises one or more β-cyclodextrin and/or adamantane moieties, optionally thiolated adamantane moieties, and the phototunable hydrogel is a viscoelastic phototunable hydrogel. In some embodiments, the phototunable hydrogel comprises a plurality of β-cyclodextrin moieties and a plurality of thiolated adamantane moieties, and at least a subset of the β-cyclodextrin moieties and the thiolated adamantane moieties form supramolecular guest-host interactions in order to confer viscosity to the phototunable hydrogel. In some embodiments, the phototunable hydrogel has a Young's modulus of less than about 5 kPa, optionally of about 0.5-1.0 kPa. In some embodiments, the phototunable hydrogel has a Young's modulus of about at least about 5 kPa, optionally of at least about 10 kPa, further optionally of at least about 15 kPa. In some embodiments, the RGD peptide comprises, consists essentially of, or consists of the amino acid sequence GCGYGRGDSPG (SEQ ID NO: 3).

In some embodiments, the presently disclosed subject matter also relates to methods for treating wounds and/or injuries in subjects in need thereof. In some embodiments, the methods comprise, consist essentially of, or consist of administering to a site of a wound or injury an effective amount of a composition comprising a phototunable hydrogel as disclosed herein; and exposing the composition to a photoinitiator, optionally lithium acylphosphinate, and a light source in an amount and for a time sufficient to cure the phototunable hydrogel at the site of the wound or injury, wherein the presence of the cured phototunable hydrogel at the site of the wound or injury enhances recovery of the wound or injury to thereby treat the wound or injury in the subject. In some embodiments, the wound is a superficial wound or injury and the composition is administered topically and then exposed to the light source. In some embodiments, the wound is an internal wound or injury and the composition comprising the phototunable hydrogel of any one of claims 1-9 is administered by injection and then exposed to the light source at the site of the internal wound or injury. In some embodiments, the internal wound or injury is a muscle injury. In some embodiments, the methods further comprise inserting a physical barrier around the site of the internal wound or injury prior to administering the composition, wherein the physical barrier retains the administered composition at the site of the internal wound or injury for at least a time before the phototunable hydrogel is cured at the site of the wound or injury. In some embodiments, the light source provides a light wavelength of about 365-505 nm, a power density of about 2-15 mW/cm², or both. In some embodiments, the exposing step is for a duration of about 2-10 minutes. In some embodiments, the cured phototunable hydrogel inhibits myofibroblast formation at the site of the wound or injury.

In some embodiments, the presently disclosed subject matter also relates to methods for inhibiting formation of scar tissue at a wound site of a subject in need thereof. In some embodiments, the methods comprise, consist essentially of, or consist of administering to the wound site an effective amount of a composition comprising a phototunable hydrogel as disclosed herein; and exposing the composition to a photoinitiator, optionally lithium acylphosphinate, and a light source in an amount and for a time sufficient to cure the phototunable hydrogel at the wound site, wherein the presence of the cured phototunable hydrogel at the wound site inhibits formation of scar tissue at the wound site.

In some embodiments, the presently disclosed subject matter also relates to methods for inhibiting fibrosis in a subject in need thereof. In some embodiments, the methods comprise, consist essentially of, or consist of administering to a site expected to undergo fibrosis in the subject an effective amount of a composition comprising a phototunable hydrogel as disclosed herein; and exposing the composition to a photoinitiator, optionally lithium acylphosphinate, and a light source in an amount and for a time sufficient to cure the phototunable hydrogel at the site expected to undergo fibrosis, wherein the presence of the cured phototunable hydrogel at the site expected to undergo fibrosis inhibits fibrosis in the subject.

In some embodiments, the presently disclosed subject matter also relates to methods for inhibiting lung fibrosis and/or scarring in a subject in need thereof. In some embodiments, the methods comprise, consist essentially of, or consist of administering to a site in a lung of the subject an effective amount of a composition comprising a phototunable hydrogel as disclosed herein; and exposing the composition to a photoinitiator, optionally lithium acylphosphinate, and a light source in an amount and for a time sufficient to cure the phototunable hydrogel at the site in the lung, whereby presence of the cured phototunable hydrogel at the site in the lung inhibits formation of lung fibrosis and/or scarring in the subject.

In some embodiments of the presently disclosed methods, the subject is a mammal, optionally a mouse or a human.

In some embodiments of the presently disclosed methods, the methods further comprise providing a photomask to at least a part of the site to provide spatiotemporal control of where covalent and/or supramolecular crosslinks occur in the hydrogel at the site.

In some embodiments of the presently disclosed methods, the administering step is repeated one or more times.

In some embodiments, the presently disclosed subject matter also relates to methods for inhibiting formation of a myofibroblast from a fibroblast, In some embodiments, the methods comprise, consist essentially of, or consist of contacting the fibroblast with an effective amount of a composition comprising a phototunable hydrogel as disclosed herein; and exposing the composition to a photoinitiator, optionally lithium acylphosphinate, and a light source in an amount and for a time sufficient to cure the phototunable hydrogel, whereby the presence of the cured phototunable hydrogel inhibits formation of a myofibroblast from the fibroblast. In some embodiments, the fibroblast is present within a subject, optionally a human.

In some embodiments, the presently disclosed subject matter also relates to methods for inhibiting expression of α-smooth muscle actin (α-SMA) and/or type I collagen in a fibroblast. In some embodiments, the methods comprise, consist essentially of, or consist of contacting the fibroblast with an effective amount of a composition comprising a phototunable hydrogel as disclosed herein; and exposing the composition to a photoinitiator, optionally lithium acylphosphinate, and a light source in an amount and for a time sufficient to cure the phototunable hydrogel, whereby the presence of the cured phototunable hydrogel inhibits expression of α-smooth muscle actin (α-SMA) and/or type I collagen in the fibroblast. In some embodiments, the fibroblast is present within a subject, optionally a human.

In some embodiments of the presently disclosed methods, the phototunable hydrogel is a soft viscoelastic hydrogel functionalized with one or more Fn9*10 fibronectin fragments.

In some embodiments of the presently disclosed methods, the phototunable hydrogel comprises a plurality of norbornene moieties, at least two of which are crosslinked to each other, optionally with a dithiol crosslinker, further optionally wherein the dithiol crosslinker comprises a covalent crosslink that results from light-mediated thiol-ene addition. In some embodiments, at least one dithiol crosslinker comprises an enzymatically-degradable peptide to thereby allow the phototunable hydrogel to degrade over time.

Accordingly, it is an object of the presently disclosed subject matter to provide compositions comprising phototunable hydrogels and methods for using the same to treat wounds and/or injury in subjects and/or to inhibit formation of scar tissue and/or fibrosis.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1. 1H NMR spectrum of norbornene-functionalized hyaluronic acid (NorHA). The degree of modification, based on norbornene peaks (“a”) relative to the methyl peak (“b”), was determined to be 31%.

FIG. 2. 1H NMR spectrum of β-cyclodextrin-functionalized hyaluronic acid (CD-HA). The degree of modification was determined to be 28%.

FIG. 3. MALDI spectrum of adamantane (Ad) peptide with the sequence 1-adamantaneacetic acid-KKKCG (SEQ ID NO: 1). Expected mass: 738.6 g/mol. Actual mass: 738.4 g/mol.

FIG. 4. Schematic of elastic and viscoelastic hyaluronic acid hydrogel design. Covalent crosslinks between norbornenes and di-thiol crosslinkers are formed via light-mediated thiol-ene addition to create elastic hydrogel networks. A combination of covalent crosslinking and supramolecular guest-host interactions between cyclodextrins and thiolated adamantane groups confer viscous characteristics to the viscoelastic system. Thiolated adhesive ligands (RGD or Fn fragments) were also incorporated during hydrogel formation.

FIGS. 5A-5D. Mechanical characterization of viscoelastic hydrogels. (FIG. 5A) Average values of soft elastic and soft viscoelastic storage (G′, white bars) and loss (G″, hatched bars) moduli measured at a constant frequency (1 Hz) and strain (1%), characterized by oscillatory shear rheology, show clear differences in loss moduli between elastic and viscoelastic groups but no significant differences as a function of adhesive ligand type (RGD, Fn9*10, and Fn4G, respectively, from left to right in each group of three bars). (FIG. 5B) Average values of stiff elastic and stiff viscoelastic storage (G′, white bars) and loss (G″, hatched bars) moduli measured at a constant frequency (1 Hz) and strain (1%), characterized by oscillatory shear rheology, show similar trends to the soft hydrogel groups (RGD, Fn9*10, and Fn4G, respectively, from left to right in each group of three bars). (FIG. 5C) Box and whisker plots of soft elastic and soft viscoelastic Young's moduli of swollen hydrogels, characterized via nanoindentation, demonstrate equivalent Young's moduli (stiffnesses) for all groups. (FIG. 5D) Box and whisker plots of stiff elastic and stiff viscoelastic Young's moduli of swollen hydrogels, characterized via nanoindentation, show similar trends to the soft hydrogel groups. Box plots of indentation data show median (horizontal line), mean (filled black circle), and have error bars corresponding to the lower value of either 1.5*interquartile range or the maximum/minimum value. At least 3 hydrogels were tested per experimental group.

FIGS. 6A-6D. In situ gelation of hydrogel groups. Rheological characterization of elastic (FIGS. 6A and 6B) and viscoelastic (FIGS. 6C and 6D) hydrogels representing normal (G′˜0.5 kPa, “soft”; FIGS. 6A and 6C) and fibrotic (G′˜5 kPa, “stiff”; FIGS. 6B and 6D) tissue. After UV light exposure, viscoelastic groups displayed loss moduli (G″) within an order of magnitude of the storage moduli (G′). The gray shaded regions show the 2 minute UV light exposures during gelation. 3 hydrogels were tested per experimental group. RGD: solid circles; Fn9*10: asterisks; Fn4G: diamonds.

FIGS. 7A-7D. Frequency-dependent behavior of viscoelastic hydrogels. (FIG. 7A) Elastic hydrogels showed frequency-independent behavior, with storage (G′) and loss moduli (G″) remaining relatively constant. (FIG. 7B) In contrast, viscoelastic hydrogels displayed frequency-dependent behavior with increasing loss moduli at increasing frequencies. (FIG. 7C) Loss tangent (tan δ) values, which represent the ratio of viscous to elastic mechanical properties (G″/G′), remained relatively constant and close to 0 for all elastic hydrogels. (FIG. 7D) In contrast, loss tangent values were elevated for viscoelastic groups across all frequencies tested and increased at higher frequencies. Similar trends were seen for the stiff groups (FIGS. 8A-8D below). Similar results for swollen hydrogel samples were measured using dynamic mechanical analysis (DMA)-like nanoindentation (FIG. 9 below). 3 hydrogels were tested per experimental group. RGD: solid circles; Fn9*10: asterisks; Fn4G: diamonds.

FIGS. 8A-8D. Rheological behavior of stiff hydrogel groups. (FIG. 8A) Stiff elastic hydrogels showed frequency-independent behavior with constant loss moduli relative to frequency. (FIG. 8B) Viscoelastic hydrogels displayed frequency-dependent behavior with increasing loss moduli at increasing frequencies. (FIG. 8C) Loss tangent (tan δ, G″/G′) values remained relatively constant for all stiff elastic hydrogels. (FIG. 8D) In contrast, loss tangent values were elevated for viscoelastic groups across all frequencies tested and increased at higher frequencies. The soft hydrogel groups can be found in FIGS. 7A-7D. RGD: solid circles; Fn9*10: asterisks; Fn4G: diamonds.

FIG. 9. Mechanical characterization of swollen hydrogels via nanoindentation. Dynamic mechanical analysis (DMA)-like analysis of PBS-swollen hydrogel groups showed similar frequency-dependent behavior for viscoelastic groups and relatively constant trends for elastic hydrogels. RGD: solid circles; Fn9*10: asterisks; Fn4G: diamonds.

FIG. 10. Stress relaxation and recovery tests. Cyclic stress relaxation and recovery tests showed full recovery of mechanical properties of hydrogel groups with stress relaxation only occurring in the viscoelastic groups for all ligand types. Strain cycled between 5% strain (times of each cycle as indicated) and 0.1% strain. RGD: thicker solid lines; Fn9*10: dashed lines: Fn4G: thinner solid lines.

FIG. 11. Fibronectin fragment-functionalized hydrogels support equivalent fibroblast attachment to RGD-modified hydrogels. Nuclei counts of fibroblasts adhered to all hydrogel experimental groups after one day showed no significant differences between RGD (1 mM) and Fn fragment (2 μM) groups. Nuclei counts were normalized to the RGD groups for each graph. 5 hydrogels were tested per experimental group. N.S.: not significant.

FIGS. 12A-12C. Fibroblast spreading is influenced by both viscoelastic mechanics and adhesive ligand type. (FIG. 12A) Human lung fibroblasts were cultured for 3 days on soft or stiff elastic and viscoelastic hydrogel groups modified with either RGD or fibronectin fragments preferentially engaging α5β1 or αvβ3. Lighter gray area correspond to areas of α-SMA expression. Brighter areas within the gray areas correspond to nuclei. (FIG. 12B) Fibroblasts preferentially binding αvβ3 (RGD, Fn4G) displayed increased spread area on elastic groups regardless of stiffness, but viscoelasticity suppressed spreading on all groups. (FIG. 12C) Cell shape index showed correlative results with spreading as smaller fibroblasts remained elongated (lower cell shape index) while larger fibroblasts assumed a more spread, activated morphology. Box plots of single cell data show median (line), mean (filled black circle), and have error bars corresponding to the lower value of either 1.5*interquartile range or the maximum/minimum value, with data points outside the 1.5*interquartile range shown as open circles. Scale bars: 100 μm, *: p<0.05; **: p<0.01; ***: p<0.001. 3 hydrogels were tested per experimental group (50-600 cells total).

FIG. 13. Nanoindentation measurements of cell stiffness. Fibroblasts are stiffer on hydrogels promoting αvβ3 engagement on stiff elastic substrates.*: p<0.05, ***: p<0.001; n=9-19 cells from 3 hydrogels per experimental group. VE: viscoelastic.

FIGS. 14A and 14B. Qualitative analysis of F-actin stress fiber and focal adhesion organization. (FIG. 14A) Percentage of human lung fibroblasts showing various levels of F-actin stress fiber organization as indicated by the representative images. More F-actin stress fibers were observed in fibroblasts on stiff elastic hydrogels, especially for groups preferentially binding αvβ3 (RGD, Fn4G) while viscoelasticity suppressed stress fiber formation across all ligand groups. Scale bars: 100 μm. 3 hydrogels were tested per experimental group (60-110 cells total). (FIG. 14B) Percentage of human lung fibroblasts showing various levels of paxillin organization as indicated by the representative images. Similarly to the results in FIG. 14A, more punctate paxillin staining was observed in fibroblasts on stiff elastic hydrogels, especially for groups preferentially binding αvβ3 (RGD, Fn4G) while viscoelasticity suppressed focal adhesion maturation across all ligand groups. Scale bars: 50 μm. 3 hydrogels were tested per experimental group (40-130 cells total). VE: viscoelastic.

FIG. 15. Qualitative analysis of α-SMA stress fiber organization. Percentage of human lung fibroblasts showing various levels of α-SMA stress fiber organization as indicated by the representative images. Scale bars: 100 μm. 3 hydrogels were tested per experimental group (60-450 cells total). VE: viscoelastic.

FIGS. 16A-16C. Preferential αvβ3 integrin engagement promotes larger focal adhesion formation. (FIG. 16A) Human lung fibroblasts seeded on hydrogels preferentially binding αvβ3 displayed more punctate paxillin staining on stiff elastic substrates, but viscoelasticity suppressed focal adhesion organization and maturation. Scale bars: 50 μm. (FIG. 16B) Ridgeline plots of focal adhesion length (determined via quantification of paxillin staining) for fibroblasts cultured on hydrogels for one day. Plots are grouped by ligand and superimposed to show variance as a function of stiffness and viscoelasticity. (FIG. 16C) The percentages of focal adhesion lengths over 1.5 μm for each hydrogel group. Fibroblasts on Fn9*10-functionalized α5β1-engaging hydrogels had smaller focal adhesions regardless of stiffness and viscoelasticity. *: p<0.05, **: p<0.01, ***: p<0.001; n>180 adhesions from at least 3 hydrogels per experimental group. VE: viscoelastic.

FIGS. 17A and 17B. Focal adhesion area quantification. (FIG. 17A) Human lung fibroblasts on hydrogels preferentially engaging αvβ3 (RGD, Fn4G) displayed increased focal adhesion area as measured by paxillin staining on stiffer, more elastic substrates while fibroblasts on Fn9*10 show reduced focal adhesion size regardless of substrate stiffness or viscoelasticity. (FIG. 17B) Focal adhesion aspect ratio quantification showed similar trends to area measurements. Box plots of single cell data show median (line), mean (filled black circle), and have error bars corresponding to the lower value of either 1.5*interquartile range or the maximum/minimum value, with data points outside the 1.5*interquartile range shown as open circles. *: p<0.05, **: p<0.01, ***: p<0.001; n>180 adhesions from at least 3 hydrogels per experimental group.

### BRIEF DESCRIPTION OF THE SEQUENCE LISTING

SEQ ID NO: 1 is the amino acid sequence of an adamantane peptide.

SEQ ID NO: 2 is the amino acid sequence of a fibronectin synergy site.

SEQ ID NO: 3 is the amino acid sequence of an exemplary RGD peptide.

SEQ ID NO: 4 is an exemplary human fibronectin amino acid sequence, in which amino acid residues 1357-1446 correspond to FNIII9 and amino acids 1447-1536 correspond to FNIII10.

### DETAILED DESCRIPTION

### I. General Considerations

Tissue fibrosis is characterized by progressive extracellular matrix (ECM) stiffening and loss of viscoelasticity that ultimately impairs organ functionality. Cells bind to the ECM through integrins, where αv integrin engagement in particular has been correlated with fibroblast activation into contractile myofibroblasts that drive fibrosis progression. There is a significant unmet need for in vitro hydrogel systems that deconstruct the complexity of native tissues to better understand the individual and combined effects of stiffness, viscoelasticity, and integrin engagement on fibroblast behavior.

Disclosed herein is the development of hyaluronic acid hydrogels with independently tunable cell-instructive properties (stiffness, viscoelasticity, ligand presentation) to address this challenge. Hydrogels with mechanics matching normal or fibrotic lung tissue were synthesized using a combination of covalent crosslinks and supramolecular interactions to tune viscoelasticity. Cell adhesion was mediated through incorporation of either RGD peptide or engineered fibronectin fragments promoting preferential integrin engagement via αvβ3 or α5β1.

Described herein are demonstrations that on fibrosis-mimicking stiff elastic hydrogels, preferential αvβ3 engagement promoted increased spreading, actin stress fiber organization, and focal adhesion maturation as indicated by paxillin organization in human lung fibroblasts. In contrast, preferential α5β1 binding suppressed these metrics. Viscoelasticity, mimicking the mechanics of healthy tissue, largely curtailed fibroblast spreading and focal adhesion organization independent of adhesive ligand type, highlighting its role in reducing fibroblast-activating behaviors. Conclusions: Together these results provide new insights into how mechanical and adhesive cues collectively guide disease-relevant cell behaviors.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As use herein, the terms “administration of” and/or “administering” a compound or composition can be understood to refer to providing a compound or composition (e.g., targeted liposomes comprising an active agent, such as a drug) of the presently disclosed subject matter to a subject in need of treatment. As used herein “administering” includes administration of a compound or composition by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, an “effective amount” or “therapeutically effective amount” refers to an amount of a compound or composition sufficient to produce a selected effect, such as but not limited to alleviating symptoms of a condition, disease, or disorder. In the context of administering a compound or composition in the form of a combination, such as multiple compositions, the amount of each a compound or composition, when administered in combination with one or more other compositions, may be different from when that composition is administered alone. Thus, an effective amount of a combination of compounds or compositions refers collectively to the combination as a whole, although the actual amounts of each a compound or composition may vary. The term “more effective” means that the selected effect occurs to a greater extent by one treatment relative to the second treatment to which it is being compared.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

The methods and compositions disclosed herein can be used on a sample either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

As used herein, amino acids are represented by the full name thereof, by the three letter code corresponding thereto, and/or by the one-letter code corresponding thereto, as summarized in Table 1.

TABLE 1
Amino Acids and Codes Therefor

| | 3-Letter | 1-Letter | | 3-Letter | 1-Letter |
|---|---|---|---|---|---|
| Full Name | Code | Code | Full Name | Code | Code |
| Aspartic Acid | Asp | D | Threonine | Thr | T |
| Glutamic Acid | Glu | E | Glycine | Gly | G |
| Lysine | Lys | K | Alanine | Ala | A |
| Arginine | Arg | R | Valine | Val | V |
| Histidine | His | H | Leucine | Leu | L |
| Tyrosine | Tyr | Y | Isoleucine | Ile | I |
| Cysteine | Cys | C | Methionine | Met | M |
| Asparagine | Asn | N | Proline | Pro | P |
| Glutamine | Gln | Q | Phenylalanine | Phe | F |
| Serine | Ser | S | Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

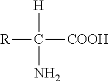

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide, and can include recombinant polypeptides. Synthetic peptides or polypeptides can be synthesized, for example, using an automated polypeptide synthesizer. Various solid phase peptide synthesis methods are known to those of skill in the art.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

### III. Compositions

### III.A. Generally

In some embodiments, the presently disclosed subject matter relates to phototunable hydrogels. As used herein, the term “phototunable” refers to a composition, such as but not limited to a hydrogel, to have at least one property (e.g., stiffness, viscoelasticity, and/or ligand presentation) that is modifiable and/or alterable using light.

In some embodiments, the phototunable hydrogels of the presently disclosed subject matter comprise, consist essentially of, or consist of backbone, which in some embodiments is a norbornene-functionalized hyaluronic acid (HA) backbone.

In some embodiments, the one or more additional functional moieties are designed to interact with each other, and in some embodiments these interactions are phototunable. Exemplary interactions include covalent interactions including but not limited to inter- and/or intramolecular crosslinking. In some embodiments, at least two norbornene moieties present on the norbornene-functionalized HA backbone are crosslinked to each other, optionally with a dithiol crosslinker. In some embodiments, the dithiol crosslinker comprises a covalent crosslink that results from light-mediated thiol-ene addition.

In some embodiments, the norbornene-functionalized HA backbone is further functionalized with one or more peptides and/or polypeptide fragments attached thereto. In some embodiments, the one or more peptides and/or polypeptide fragments are selected from the group consisting of an RGD peptide and a fibronectin (Fn) polypeptide fragment (i.e., not a full length fibronectin amino acid sequence but a subsequence thereof). Subsequences of Fn are known, including several that have well characterized biological activities and binding activities. Examples of Fn subsequences include, but are not limited to an Fn α5β1 peptide and an Fn αvβ3 peptide. In some embodiments, one or more Fn subsequences are thiolated, optionally at the N-terminus. In some embodiments, combinations of Fn fragments are employed in the hydrogels of the presently disclosed subject matter.

In some embodiments, a phototunable hydrogel of the presently disclosed subject matter includes an RGD peptide. In some embodiments, the RGD peptide comprises, consists essentially of, or consists of the amino acid sequence GCGYGRGDSPG (SEQ ID NO: 3).

The HA backbone of the presently disclosed hydrogels can also be functionalized with one or more additional functional moieties, such as but not limited to β-cyclodextrin and adamantane. As is known, β-cyclodextrin and adamantane can interact with each other to form in some embodiments supramolecular guest-host interactions. These supramolecular guest-host interactions can also influence the properties of the presently disclosed hydrogels, and thus in some embodiments concentrations of β-cyclodextrin and/or adamantane in the hydrogels can be modified in order to provide desirable properties. By way of example and not limitation, in some embodiments a phototunable hydrogel of the presently disclosed subject matter comprises a plurality of β-cyclodextrin moieties and a plurality of thiolated adamantane moieties, and at least a subset of the β-cyclodextrin moieties and the thiolated adamantane moieties form supramolecular guest-host interactions in order to confer viscosity to the phototunable hydrogel.

As disclosed herein, the designs of the hydrogels of the presently disclosed subject matter can be manipulated in order to prepare elastic phototunable hydrogels and viscoelastic phototunable hydrogels. By way of example and not limitation, in some embodiments the norbornene-functionalized HA backbone of a hydrogel of the presently disclosed subject matter lacks β-cyclodextrin and adamantane, which can result in the phototunable hydrogel being an elastic phototunable hydrogel. Alternatively, in some embodiments the norbornene-functionalized HA backbone comprises one or more β-cyclodextrin and one or more adamantane moieties, optionally thiolated adamantane moieties, which can result in the phototunable hydrogel being a viscoelastic phototunable hydrogel.

In some embodiments, a phototunable hydrogel of the presently disclosed subject matter is designed to have a Young's modulus of less than about 5 kPa, optionally of about 0.5-1.0 kPa. Such phototunable hydrogels can mimic normal cells and tissues. In some embodiments, however, a phototunable hydrogel of the presently disclosed subject matter is designed to have a Young's modulus of about at least about 5 kPa, optionally of at least about 10 kPa, further optionally of at least about 15 kPa. Such phototunable hydrogels can mimic cells and tissues that have been injured, such as but not limited to scarring and/or fibrosis.

### III.B. Formulations

The compositions of the presently disclosed subject matter can be administered in any formulation or route that would be expected to deliver the compositions to whatever target site might be appropriate.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

### III.C. Dosages

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in scarring and/or fibrosis, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, a pharmaceutically or therapeutically effective amount of a phototunable hydrogel of the presently disclosed subject matter is administered to a subject at a site of a wound and/or injury, and/or at a site where fibrosis is and/or might occur, and/or at a site where transition of fibroblasts to myofibroblasts would be undesirable.

### III.D. Routes of Administration

Suitable methods for administration of the compositions of the presently disclosed subject matter include, but are not limited to intravenous administration, oral delivery, and delivery directly to a target tissue or organ (e.g., a topical application and/or a site of injury such as but not limited to a muscle injury). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sufobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of a liquid, suspension, cream, ointment, lotion, or the like, preferably in unit dosage form suitable for single administration of a precise dosage. The compositions can in some embodiments include one or more pharmaceutically acceptable carriers and, in addition, may include other pharmaceutical agents, adjuvants, diluents, buffers, etc.

In some embodiments, the mode of administration is a liquid form, which can then be cured by application of light of the appropriate wavelength, intensity, and duration to cure the phototunable hydrogels of the presently disclosed subject matter at a site of interest.

### IV. Methods and Uses

The phototunable hydrogels of the presently disclosed subject matter can be employed for various in vitro, ex vivo, and/or in vivo uses. For example and as disclosed herein, the hydrogels can be employed for examining and testing the biological activities of fibroblasts as they interact with various extracellular cues such as, but not limited to, engagement with integrins αvβ3 or α5β1. Such examinations are described herein, in which spreading, actin stress fiber organization, and focal adhesion maturation as indicated by paxillin organization of fibroblasts were tested.

As a result of these investigations, several uses for compositions comprising, consisting essentially of, or consisting of the phototunable hydrogels of the presently disclosed subject matter are also described herein.

In some embodiments, the presently disclosed subject matter thus relates to methods for treating wounds and/or injuries in subjects in need thereof. In some embodiments, the methods comprise, consist essentially of, or consist of: (a) administering to a site of a wound or injury an effective amount of a composition comprising a phototunable hydrogel as described herein; and (b) exposing the composition to a photoinitiator, optionally lithium acylphosphinate, and a light source in an amount and for a time sufficient to cure the phototunable hydrogel at the site of the wound or injury, wherein the presence of the cured phototunable hydrogel at the site of the wound or injury enhances recovery of the wound or injury to thereby treat the wound or injury in the subject.

Various wounds and/or injuries can be treated with the phototunable hydrogels of the presently disclosed subject matter. In some embodiments, a wound and/or injury is a superficial wound and/or injury and the composition is administered topically. After administration to a site of injury, the phototunable hydrogel can be exposed to a light source to cure the phototunable hydrogel at the site, thereby enhancing recovery of the wound or injury to thereby treat the wound or injury in the subject.

Alternatively, in some embodiments the wound and/or injury is an internal wound and/or injury and the composition comprising the phototunable hydrogel of the presently disclosed subject matter is administered by injection to the site of the injury before being exposed to the light source at the site of the internal wound and/or injury. By way of example and not limitation, in some embodiments an internal wound and/or injury is a muscle injury, and the phototunable hydrogel of the presently disclosed subject matter enhances repair of the muscle injury.

In some embodiments, it can be desirable to limit diffusion of the phototunable hydrogel of the presently disclosed subject matter from the site of an injury before and during the exposure to light. Thus, in some embodiments, the methods of the presently disclosed subject matter can further comprise inserting a physical barrier around the site of the wound and/or injury prior to administering the composition, wherein the physical barrier retains the administered composition at the site of the wound and/or injury for at least a time before the phototunable hydrogel is acceptably cured at the site of the wound and/or injury.

Once a phototunable hydrogel of the presently disclosed subject matter is administered or otherwise delivered to the desired site, the phototunable hydrogel is exposed to light in order to cure (e.g., solidify) the phototunable hydrogel. Depending on the nature of the phototunable hydrogel and the desired use, various wavelengths and intensities of light as well as various durations of light exposure can be employed to cure the phototunable hydrogels of the presently disclosed subject matter as desired. By way of example and not limitation, in some embodiments a light source provides a light wavelength of about 365-505 nm. In some embodiments, a light source provides a power density of about 2-15 mW/cm². In some embodiments, the exposing step is for a duration of about 2-10 minutes.

As disclosed herein, one advantage of the phototunable hydrogels of the presently disclosed subject matter is that they can be designed to inhibit myofibroblast formation at the site of a wound and/or injury. This can have various beneficial outcomes including but not limited to reduction in scarring and/or reduction in formation of fibrotic lesions and focal adhesions (FAs).

Thus, in some embodiments the presently disclosed subject matter provides methods for inhibiting formation of scar tissue at a wound site of a subject in need thereof. In some embodiments, the methods comprise, consist essentially of, or consist of (a) administering to the wound site an effective amount of a composition comprising a phototunable hydrogel of the presently disclosed subject matter; and (b) exposing the composition to a photoinitiator, optionally lithium acylphosphinate, and a light source in an amount and for a time sufficient to cure the phototunable hydrogel at the wound site, wherein the presence of the cured phototunable hydrogel at the wound site inhibits formation of scar tissue at the wound site.

Similarly, in some embodiments the presently disclosed subject matter provides methods for inhibiting fibrosis in a subject in need thereof by (a) administering to a site expected to undergo fibrosis in the subject an effective amount of a composition comprising a phototunable hydrogel of the presently disclosed subject matter; and (b) exposing the composition to a photoinitiator, optionally lithium acylphosphinate, and a light source in an amount and for a time sufficient to cure the phototunable hydrogel at the site expected to undergo fibrosis, wherein the presence of the cured phototunable hydrogel at the site expected to undergo fibrosis inhibits fibrosis in the subject. In a particular embodiment, the subject's injury can lead to fibrosis and/or scarring of the lung. Therefore, in some embodiments the presently disclosed subject matter relates to (a) administering to a site in a lung of the subject an effective amount of a composition comprising a phototunable hydrogel of the presently disclosed subject matter; and (b) exposing the composition to a photoinitiator, optionally lithium acylphosphinate, and a light source in an amount and for a time sufficient to cure the phototunable hydrogel at the site in the lung, whereby presence of the cured phototunable hydrogel at the site in the lung inhibits formation of lung fibrosis and/or scarring in the subject.

In some embodiments, it is desirable to avoid exposing cells and/or tissue in the vicinity of the wound and/or injury (e.g., normal and/or unaffected cells and/or tissue) to the light source. In such cases, the presently disclosed methods can further comprise providing a photomask to at least a part of the site to provide spatiotemporal control of where covalent and/or supramolecular crosslinks occur in the hydrogel at the site. See Hui et al., 2019 for a description of using photomasks to provide spatiotemporal control of hydrogel curing.

As set forth herein, in some embodiments the presently disclosed subject matter relates to methods for inhibiting formation of myofibroblasts from fibroblasts, in some embodiments at sites at which myofibroblast formation would be undesirable. In such embodiments, the presently disclosed methods can comprise, consist essentially of, or consist of (a) contacting the fibroblast or fibroblasts with an effective amount of a composition comprising a phototunable hydrogel of the presently disclosed subject matter; and (b) exposing the composition to a photoinitiator, optionally lithium acylphosphinate, and a light source in an amount and for a time sufficient to cure the phototunable hydrogel, whereby the presence of the cured phototunable hydrogel inhibits formation of a myofibroblast from the fibroblast.

Similarly, wounds and/or injuries are known to the induce undesirable expression of fibrogenic genes encoding α-smooth muscle actin (α-SMA) and type I collagen. As such, in some embodiments the presently disclosed subject matter also relates to methods for inhibiting undesirable expression of α-smooth muscle actin (α-SMA) and/or type I collagen in fibroblasts. In some embodiments, the presently disclosed methods thus comprise, consist essentially of, or consist of (a) contacting the fibroblast with an effective amount of a composition comprising a phototunable hydrogel of the presently disclosed subject matter; and (b) exposing the composition to a photoinitiator, optionally lithium acylphosphinate, and a light source in an amount and for a time sufficient to cure the phototunable hydrogel, whereby the presence of the cured phototunable hydrogel inhibits expression of α-smooth muscle actin (α-SMA) and/or type I collagen in the fibroblast. The presently disclosed methods can be performed in vitro, ex vivo, or in vivo, and thus in some embodiments the fibroblast can be present within a subject, optionally a human.

In some embodiments, the phototunable hydrogel that is employed in the methods of the presently disclosed subject matter is a soft viscoelastic hydrogel functionalized with one or more Fn9*10 fibronectin fragments.

In some embodiments, the phototunable hydrogel that is employed in the methods of the presently disclosed subject matter comprises a plurality of norbornene moieties, at least two of which are crosslinked to each other, optionally with a dithiol crosslinker, further optionally wherein the dithiol crosslinker comprises a covalent crosslink that results from light-mediated thiol-ene addition. In some embodiments, at least one dithiol crosslinker comprises an enzymatically-degradable peptide to thereby allow the phototunable hydrogel to degrade over time.

It is understood that a desired dosage of a composition of the presently disclosed subject matter can be obtained through more than one administration of the composition. As such, in some embodiments the administering step is repeated one or more times.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the Examples

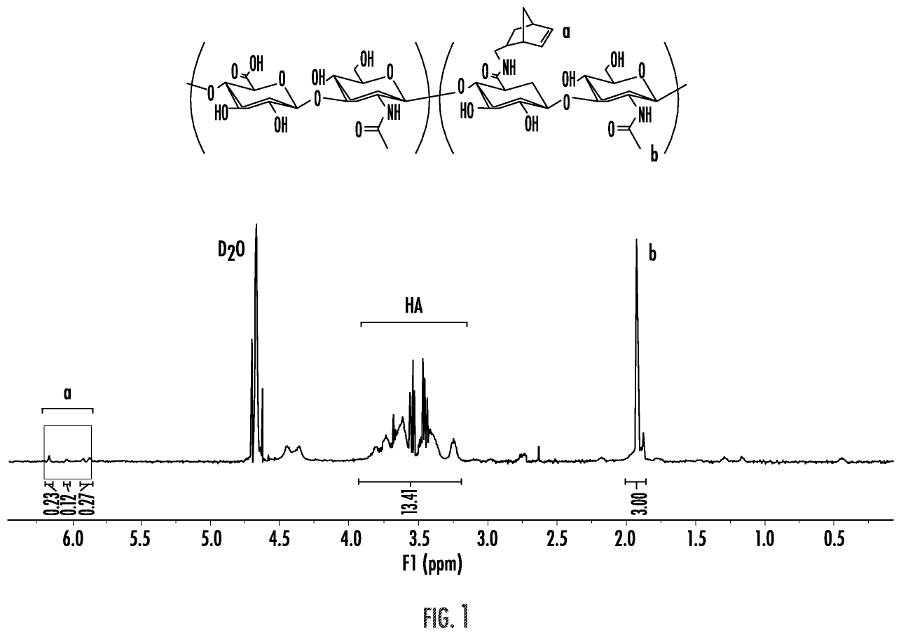

NorHA synthesis. HA was functionalized with norbornene groups as previously described (Gramlich et al., 2013; Hui et al., 2019). Sodium hyaluronate (Lifecore, 62 kDa) was converted to hyaluronic acid tert-butyl ammonium salt (HA-TBA) via proton exchange with Dowex 50 W resin prior to being filtered, titrated to pH 7.05, frozen, and lyophilized. 5-norbornene-2-methylamine and benzotriazole-1-yloxytris-(dimethylamino)phosphonium hexafluorophosphate (BOP) were added dropwise to HA-TBA in dimethylsulfoxide (DMSO) and reacted for 2 hours at 25° C., quenched with cold water, dialyzed (molecular weight cutoff: 6-8 kDa) for 5 days, filtered, dialyzed for 5 more days, frozen, and lyophilized. The degree of modification was 31% as determined via proton nuclear magnetic resonance (¹H NMR, 500 MHz Varian Inova 500 see FIG. 1).

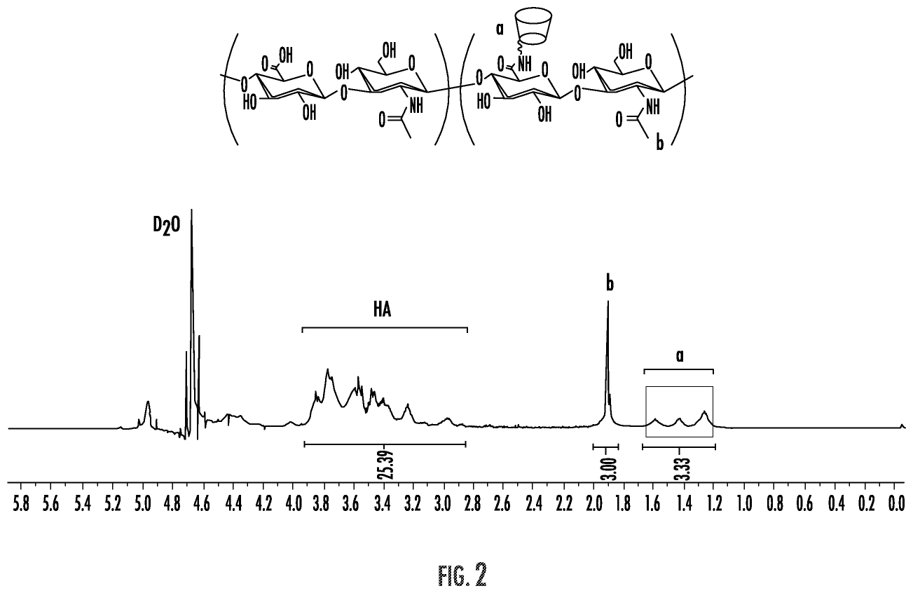

β-CD-HA synthesis. β-cyclodextrin modified hyaluronic acid (CD-HA) was synthesized by coupling synthesized 6-(6-aminohexyl)amino-6-deoxy-β-cyclodextrin (β-CD-HDA) to HA-TBA in anhydrous DMSO in the presence of BOP (Rodell et al., 2013; Hui et al., 2019). The amidation reaction was carried out at 25° C. for 3 hours, quenched with cold water, dialyzed for 5 days, filtered, dialyzed for 5 more days, frozen, and lyophilized. The degree of modification was 28% as determined by ¹H NMR (see FIG. 2).

Peptide synthesis. Thiolated adamantane peptide (Ad-KKKCG; SEQ ID NO: 1) was synthesized on Rink Amide MBHA high-loaded (0.78 mmol/g) resin using solid phase peptide synthesis as described in Hui et al., 2019. The peptide was cleaved in 95% trifluoroacetic acid, 2.5% triisopropylsilane, and 2.5% H₂O for 2-3 hours, precipitated in cold ether, dried, resuspended in water, frozen, and lyophilized. Synthesis was confirmed via matrix-assisted laser desorption/ionization (MALDI) mass spectrometry (see FIG. 3).

Recombinant fibronectin fragments. Recombinant fibronectin fragments of the ninth and tenth type III repeat units (FnIII₉ and FnIII₁₀) were designed to preferentially bind α5β1 or αvβ3 integrin heterodimers as described (Martino et al., 2009; Li et al., 2017; Fiore et al., 2018). Fibronectin fragments were separately expressed in E. coli and purified via a Strep-Tag II column in house. Briefly, to promote α5β1 binding, FnIII₉ was thermodynamically stabilized through a leucine to proline point mutation at position 1408, which has demonstrated stabilization of the spatial orientation of the RGD motif on FnIII₁₀ and the synergy site PHSRN (SEQ ID NO: 2) on FnIII₉, increasing selectivity to β1 integrins (Cao et al., 2017). While this fragment still supports αvβ3 binding, it has greater α5β1 integrin-binding affinity (K_{D}˜12 nM for α5β1 versus ˜40 nM for αvβ3; Cao et al., 2017). We have referred to this fragment as “Fn9*10” herein. For preferential αvβ3 integrin binding, four glycine residues were inserted into the liner region between FnIII₉ and FnIII₁₀ to disrupt α5β1 binding by increasing the separation between the RGD and PHSRN (SEQ ID NO: 2) sites. This fragment is referred to herein as “Fn4G”. Both fibronectin fragments contained N-terminal cysteine residues to enable thiol-ene coupling to the HA hydrogels. Fragment quality was validated using ELISA. The thiolated fragments were first covalently bound to maleimide-activated plates (Thermo Fisher Scientific, 15150, 20 μg/mL). Bound fragments were then detected using the single chain fragment antibody H5 engineered to recognize both Fn9*10 and Fn4G as described in Cao et al., 2017.

HA hydrogel fabrication. Thin film hydrogels (18×18 mm, ˜100 μm thickness) were fabricated on thiolated coverslips via ultraviolet (UV)-light mediated thiol-ene addition, similar to previously established methods (Hui et al., 2019). “Soft” and “stiff” hydrogel formulations were designed to match normal (Young's modulus or stiffness˜1 kPa) and fibrotic (˜15 kPa) stiffnesses, respectively (Liu et al., 2010; Booth et al., 2012; Wells, 2014; Fiore et al., 2018). Covalently-crosslinked soft (2 wt % NorHA) and stiff (6 wt % NorHA) elastic hydrogels formulations were crosslinked with dithiothreitol (DTT, thiol-norbornene ratios of 0.22 and 0.35 for soft and stiff groups, respectively). Soft (2 wt % NorHA-CDHA) and stiff (6 wt % NorHA-CDHA) viscoelastic hydrogels were fabricated through a combination of covalent and physical crosslinking. NorHA and DTT (covalent crosslinks, thiol-norbornene ratios of 0.35 and 0.55 for soft and stiff groups, respectively) were combined with CD-HA and thiolated adamantane (Ad) peptides (supramolecular guest-host interactions between CD and Ad, 1:1 molar ratio of CD to Ad). Cell adhesion was enabled in all hydrogel groups through incorporation of either 1 mM RGD peptide (GCGYGRGDSPG; SEQ ID NO: 3); Genscript) or 2 μM thiolated Fn fragments (Fn9*10 or Fn4G). Hydrogel solutions were photopolymerized (365 nm, 5 mW/cm²) between coverslips in the presence of 1 mM lithium acylphosphinate (LAP) photoinitiator for 2 minutes and swelled in PBS overnight at 37° C. before subsequent experiments.

Mechanical characterization. Hydrogel rheological properties were quantified on an Anton Paar MCR 302 rheometer using a cone-plate geometry (25 mm diameter, 0.5°, 25 μm gap). In situ gelation via 2 minute UV light irradiation (5 mW/cm²) was tracked using oscillatory time sweeps (1 Hz, 1% strain) followed by oscillatory frequency sweeps (0.001-10 Hz, 1% strain) and cyclic stress relaxation and recovery tests alternating between 0.1% and 5% strain. Nanoindentation tests were performed using Optics11 Piuma and Chiaro nanoindenters on hydrogels swollen in PBS for at least 24 hours to determine hydrogel mechanical characteristics. A 25 μm diameter spherical borosilicate glass probe attached to a cantilever with a spring constant of 0.5 N/m was used during testing. For each indentation, the loading portion of the generated force versus distance indentation curve was used to determine the Young's modulus by applying the Hertzian contact mechanics model and assuming a Poisson's ratio of 0.5. The Optics11 nanoindenter software also features a dynamic operational mode to enable dynamic mechanical analysis (DMA)-like measurements through mechanical oscillations. DMA measurements were performed to quantify viscoelasticity (G′ and G″) of swollen hydrogels via frequency sweeps (0.1-10 Hz) and force relaxation tests.

Cell culture. Human lung fibroblasts (hTERT T1015, abmgood) were used between passages 7-12 and culture medium was changed every 2-3 days (Gibco Dulbecco's Modified Eagle Medium (DMEM) supplemented with 10 v/v % fetal bovine serum (FBS) and 1 v/v % antibiotic antimycotic (1,000 U/mL penicillin, 1,000 μg/mL streptomycin, and 0.25 μg/mL amphotericin B)). Normal human lung fibroblasts (CC-2512, Lonza) were used between passages 3-5 for paxillin experiments and culture medium was changed every 2-3 days (Lonza FBM Basal Medium supplemented with 2 v/v % fetal bovine serum (FBS), 0.1 v/v % human recombinant insulin (1-20 μg/mL), 0.1 v/v % recombinant human fibroblast growth factor-B (rhFGF-B, 0.5-5 ng/mL), and 0.1 v/v % gentamicin sulfate amphotericin B (GA-1000, 30 μg/mL gentamicin and 15 ng/mL amphotericin)). Swelled hydrogels were sterilized in non-TC-treated 6-well plates via germicidal UV irradiation for at least 2 hours and incubated in culture medium for at least 30 minutes prior to cell seeding. Cells were seeded at 2×10⁴ cells/hydrogel (18×18 mm).

Immunostaining, imaging, and analysis. Cell-seeded hydrogels were fixed in 10% neutral-buffered formalin for 15 minutes, permeabilized in PBST (0.1% Triton X-100 in PBS) for 10 minutes, and blocked in 3% bovine serum albumin (BSA) in PBS for at least 1 hour at 25° C. To visualize focal adhesions (FAs), cells were fixed using a microtubule stabilization buffer for 10 minutes at 37° C. before blocking. Hydrogels were then incubated overnight at 4° C. with primary antibodies. Primary antibodies used in this work included paxillin (mouse monoclonal anti-paxillin B-2, Santa Cruz Biotechnology, sc-365379, 1:500) to visualize FA formation and α-smooth muscle actin (α-SMA, mouse monoclonal anti-α-SMA clone 1A4, Sigma-Aldrich, A2547, 1:400). Hydrogels were washed three times using PBS and incubated with secondary antibodies (AlexaFluor 488 goat anti-rabbit IgG or AlexaFluor 555 goat anti-mouse IgG, Invitrogen, 1:600-800) and/or rhodamine phalloidin (Invitrogen, R415 1:600) to visualize F-actin for 2 hours in the dark at 25° C. Hydrogels were rinsed three times with PBS and incubated with a DAPI nuclear stain (Invitrogen, D1306, 1:10000) for 1 minute before washing with PBS. Images were taken on a Zeiss AxioObserver 7 inverted microscope. Cell spread area and cell shape index were determined using a CellProfiler (Broad Institute, Harvard/MIT) pipeline modified to include adaptive thresholding. Cell shape index determines the circularity of the cell, where a line and a circle have values of 0 and 1, respectively, and was calculated using the formula:

$$CSI = \frac{4 π A}{{P}^{2}}$$

where A is the cell area and P is the cell perimeter. For qualitative analysis of actin stress fiber organization, cells were binned into three categories—“mostly stress fibers” showed stress fibers in over 60% of the cell area, “some stress fibers” constituted those with roughly 15-60% stress fibers, and “no stress fibers” showed only diffuse actin staining. For FA analysis, cells stained with paxillin were imaged using a 40× oil objective. FA count, area, and fluorescence intensity were quantified via the Focal Adhesion Analysis Server (FAAS; Berginski & Gomez, 2013) automated imaging processing pipeline using a 4.5 threshold and minimum pixel size of 25.

Statistical analysis. For mechanical characterization, at least 3 technical replicates were performed and the data are presented as mean±standard deviation. For statistical comparisons between hydrogel groups, two-way ANOVA with Tukey's HSD post hoc analysis (more than two experimental groups) were performed. All experiments included at least 3 replicate hydrogels per experimental group. Box plots of single cell data include median/mean indicators as well as error bars corresponding to the lower value of either the 1.5*interquartile range or the maximum/minimum value, with data points outside the 1.5*interquartile range shown as open circles. Statistically-significant differences are indicated by *, **, or *** corresponding to p<0.05, 0.01, or 0.001, respectively.

### Example 1

### Hydrogels Were Designed to Independently Control Stiffness, Viscoelasticity, and Presentation of Integrin-Binding Adhesive Sites

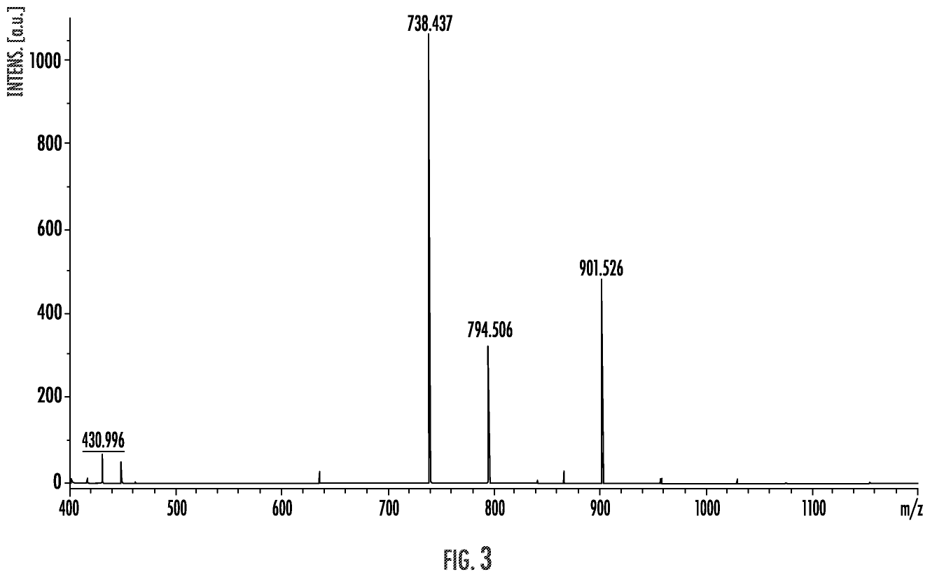

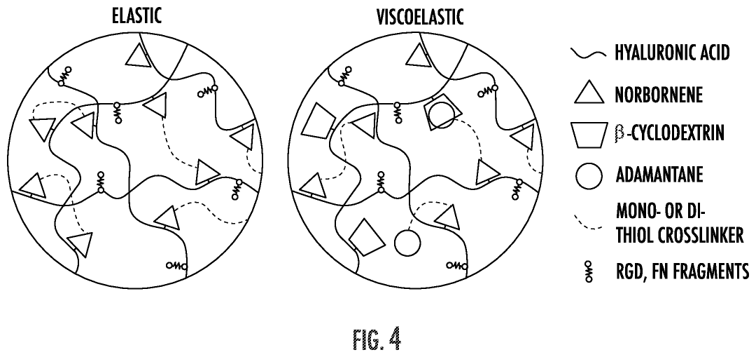

Hyaluronic acid (HA) hydrogels representing normal (G′˜0.5 kPa) and fibrotic (G′˜5 kPa) lung tissue mechanics were fabricated with a combination of covalent crosslinks and supramolecular guest-host interactions to impart viscous properties (FIG. 4; Rodell et al., 2013; Rodell et al., 2016; Hui et al., 2019). HA was chosen as the hydrogel backbone for its ability to be chemically modified with various functional groups to achieve a range of viscoelastic properties covering healthy and diseased soft tissue, as shown previously (Burdick & Prestwich, 2011; Schanté et al., 2011; Gramlich et al., 2013; Caliari et al., 2016a; Hui et al., 2019; Kwon et al., 2019). Stiffness was controlled primarily through adjusting the concentration of HA and the ratio of dithiol crosslinker to norbornene groups on HA. Several methods to incorporate viscoelasticity into material systems have been developed, including the addition of sterically entrapped high molecular weight linear polymers to introduce viscosity (Aida et al., 2012; Charrier et al., 2018), covalent adaptable networks (McKinnon et al., 2014; Tang et al., 2018; Marozas et al., 2019), physical crosslinking of natural polymers (e.g., alginate (Zhao et al., 2010; Chaudhuri et al., 2016), collagen (Mohammadi et al., 2015; Nam et al., 2016)) for modulation of stress relaxation properties, and supramolecular crosslinking chemistries (e.g., host-guest complexes; van de Manakker et al., 2010; Rodell et al., 2013; Rodell et al., 2015; Hui et al., 2019). As described herein, the addition of supramolecular guest-host interactions between β-cyclodextrin HA (CD-HA) and thiolated adamantane (Ad) peptides (1:1 molar ratio of CD to Ad), where the hydrophobic Ad guest moiety has a high affinity for the hydrophobic interior of CD, introduced viscous characteristics into the system (Rodell et al., 2013; Hui et al., 2019). Elastic hydrogel substrates contained only covalent crosslinks, while viscoelastic substrates included a combination of covalent and supramolecular interactions.

While HA is a natural ECM component and interacts with cell surface receptors including CD44 and RHAMM in its unmodified forms, it does not support integrin binding, allowing customization of these interactions in the presently disclosed hydrogel designs (Dicker et al., 2014; Caliari & Burdick, 2016). In addition to controlling hydrogel stiffness and viscoelasticity by modulating crosslinking as described above, it was hypothesized that one could also dictate cellular adhesion through the incorporation of either thiolated RGD peptide or Fn fragments designed to preferentially bind αvβ3 (Fn4G) or α5β1 (Fn9*10) integrins (Martino et al., 2009; Li et al., 2017; Fiore et al., 2018). Preferential α5β1 engagement in Fn9*10 is engineered by stabilizing the spatial proximity of the PHSRN (SEQ ID NO: 2) synergy site on FnIII₉ with the RGD on FnIII₁₀, although Fn9*10 can also bind αvβ3 (Cao et al., 2017). Insertion of a four glycine spacer between FnIII₉ and FnIII₁₀ in Fn4G abrogates simultaneous binding to both the PHSRN (SEQ ID NO: 2) and RGD sequences necessary for α5β1 engagement, leading to preferential αvβ3 binding (Cao et al., 2017). Since the RGD peptide does not contain the PHSRN (SEQ ID NO: 2) synergy sequence, it was anticipated that it would also preferentially engage αvβ3 over α5β1. Overall, the modular hydrogel design allows independent control of HA content, crosslinking type and density, and adhesive ligand incorporation to enable simultaneous tuning of stiffness, viscoelasticity, and integrin engagement.

### Example 2

### Incorporation of Fibronectin-Related Adhesive Ligands Did Not Impact Hydrogel Mechanics

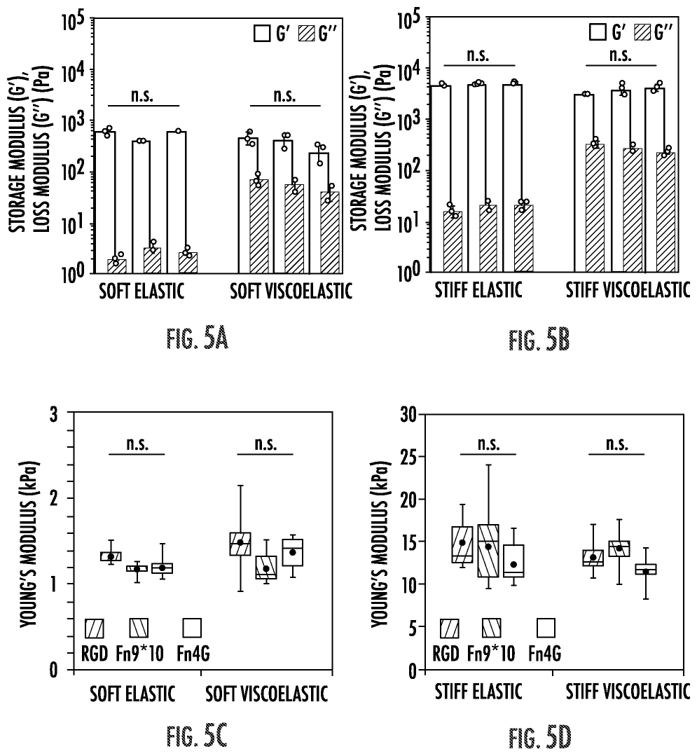

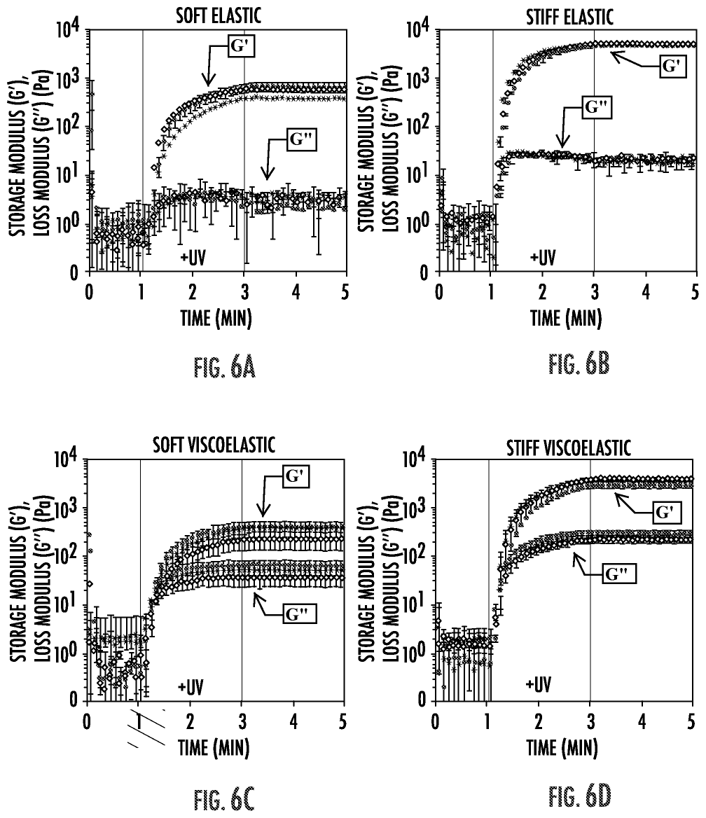

To determine if incorporating different adhesive ligands would impact the ability to independently control hydrogel stiffness and viscoelasticity was also tested. Hydrogel mechanics were examined through in situ oscillatory shear rheology (FIGS. 5A and 5B) and nanoindentation of PBS-swollen hydrogels (FIGS. 5C and 5D). Rapid in situ gelation for all hydrogel experimental groups was confirmed via rheology (FIGS. 6A-6D). The introduction of fibronectin-based adhesive ligands did not affect overall mechanics; similar storage and loss moduli were observed for all groups compared to RGD-containing hydrogels. Target mechanical values for “soft” and “stiff” groups corresponding to normal (elastic modulus, E˜1 kPa) and fibrotic (E˜15 kPa) lung tissue were successfully reached. As expected, the viscoelastic hydrogel design led to increased viscous properties as evidenced by higher loss moduli (G″) that were within an order of magnitude of the storage moduli (G′), analogous to normal soft tissue like lung and liver (Perepelyuk et al., 2016).

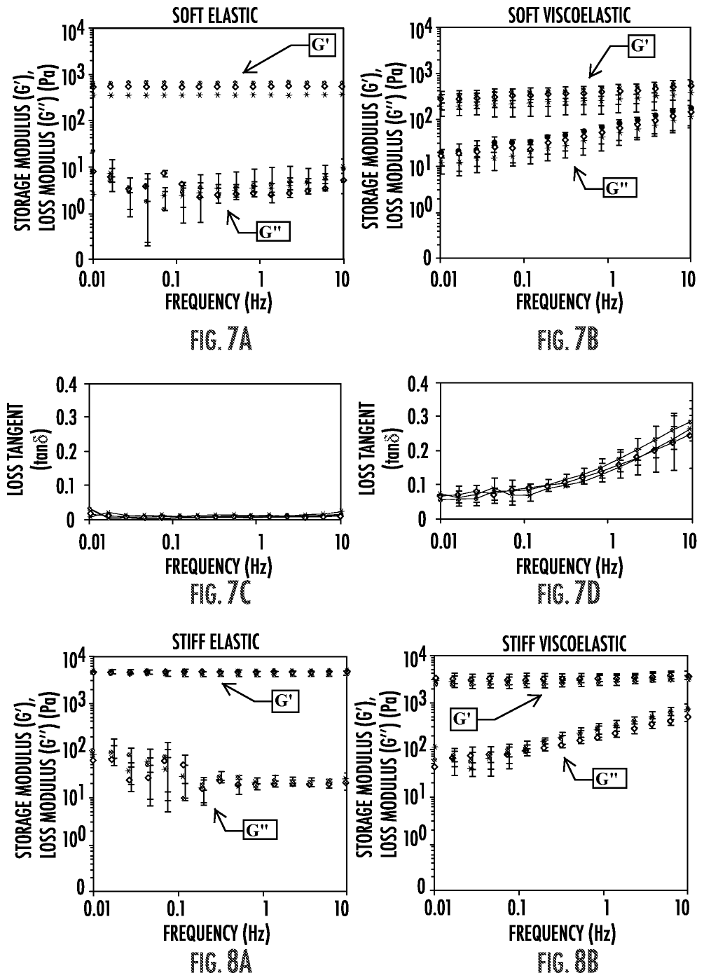

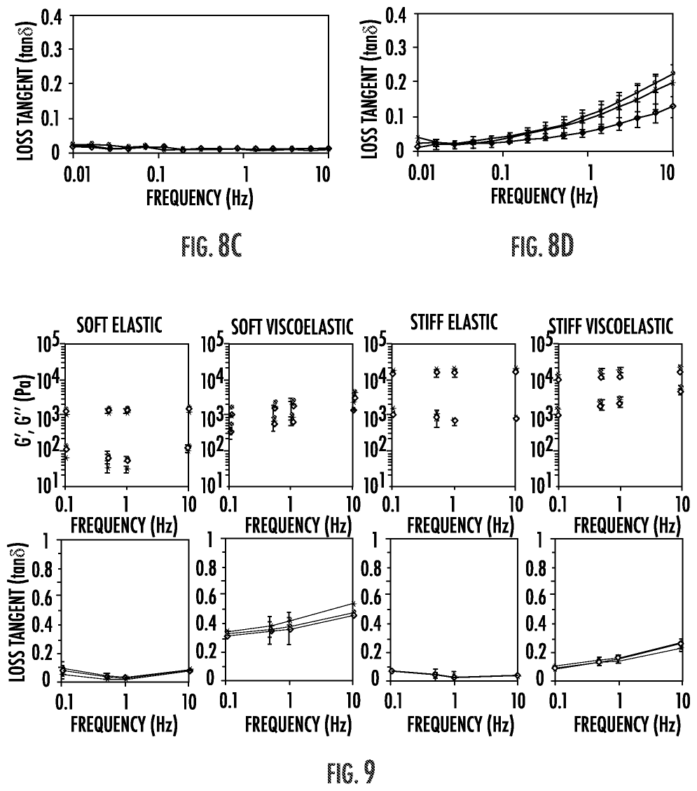

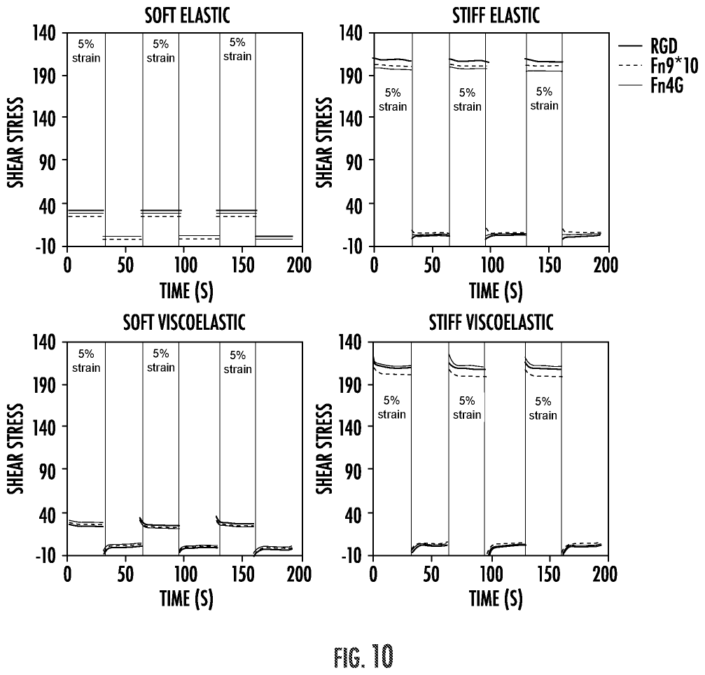

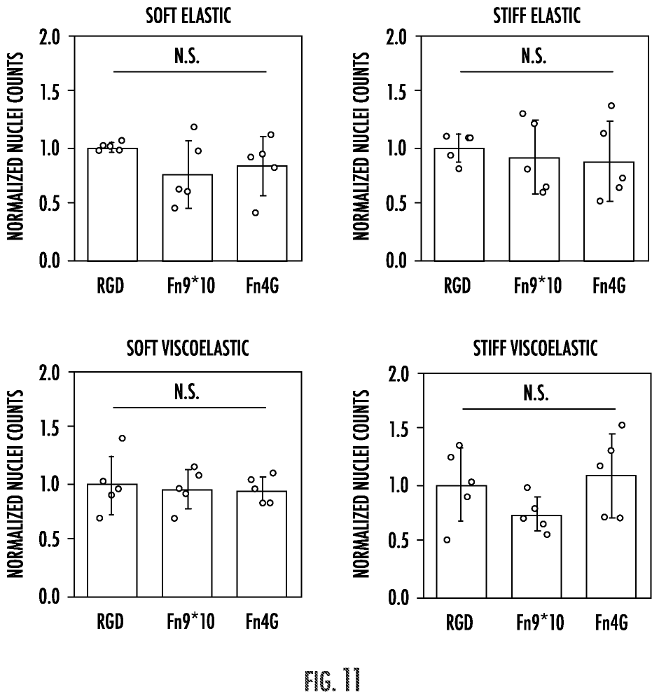

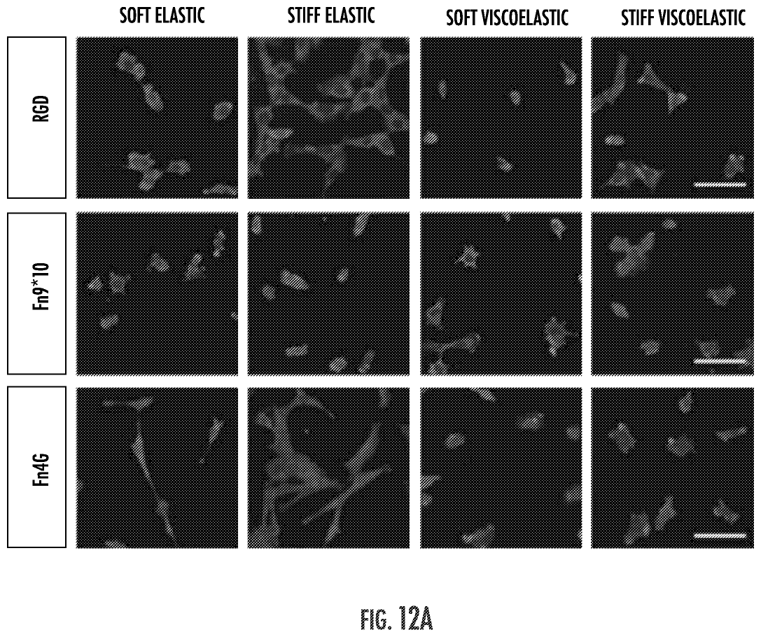

Viscoelastic substrates also displayed tissue-relevant frequency-dependent mechanical responses as measured by both rheology (FIGS. 7A-7D and 8A-8D) and DMA-like nanoindentation measurements (FIG. 9); at lower frequencies (longer time scales), the ability for guest-host interactions to re-organize and re-associate resulted in more solid-like behavior, whereas at higher frequencies (shorter time scales) guest-host interactions were disrupted with less time for complex reformation (Rodell et al., 2013; Hui et al., 2019). Stress relaxation, a key feature of viscoelastic materials, was demonstrated by observation of time-dependent decreases in storage moduli only in viscoelastic substrates when constant strain (5%) was applied (FIG. 10). The frequency-dependent relaxation behavior observed for the viscoelastic groups relates to cell-relevant time scales; cells are able to respond to force oscillations and exert traction forces on the order of seconds to minutes at a frequency of around 0.1-1 Hz (Cameron et al., 2011; Chaudhuri et al., 2016; Charrier et al., 2018). Elastic hydrogels consisting of only stable covalent crosslinks did not display stress relaxation over time.

### Example 3

### Fibroblast Spreading is Influenced by Both Viscoelasticity and Adhesive Ligand Type

After validating that hydrogels incorporating different adhesive ligands could be synthesized in both elastic and viscoelastic forms with overall stiffness matching normal and fibrotic tissue, we sought to confirm that the presently disclosed hydrogel formulations would support equivalent cell adhesion. The number of human lung fibroblasts attached to the hydrogels were quantified after one day and it was confirmed that all formulations supported similar levels of adhesion (FIG. 11). Notably, hydrogels containing only 2 μM Fn fragments allowed equivalent fibroblast attachment to hydrogels with 1 mM RGD peptide. Previous work using these fragments has also shown robust cell attachment using concentrations of this magnitude (Cao et al., 2017; Li et al., 2017). In contrast, short linear RGD peptides have previously been shown to be around 1000 times less effective in cell attachment compared to fibronectin (Hautanen et al., 1989).

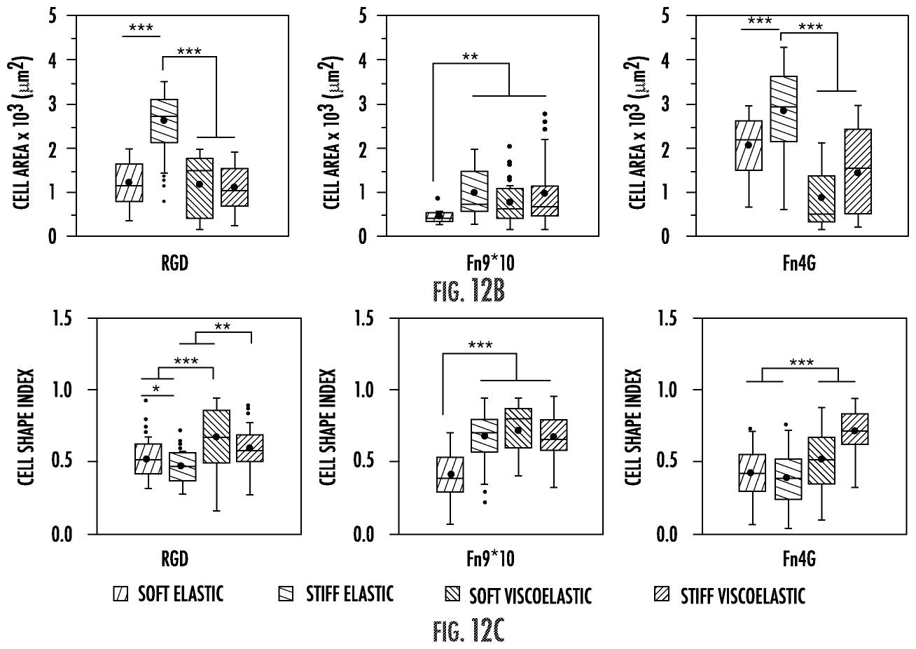

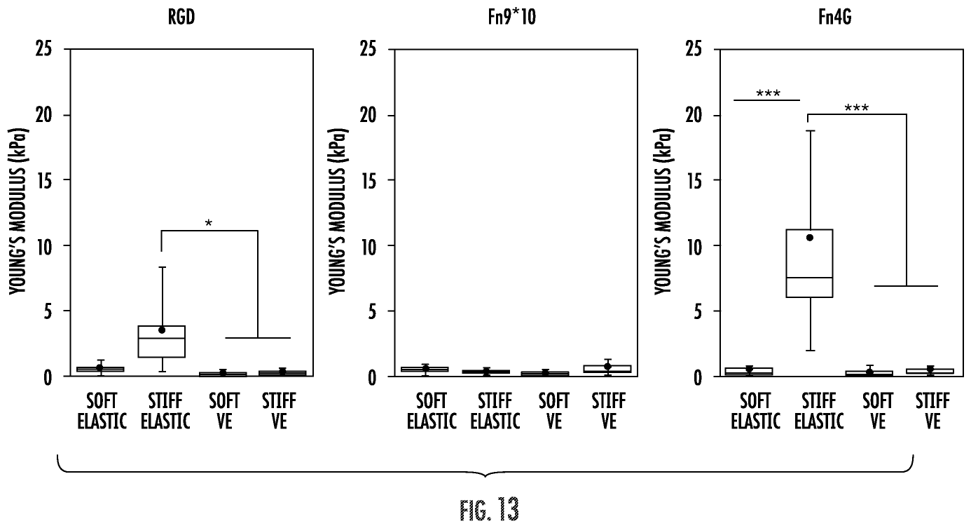

The combined influence of stiffness, viscoelasticity, and adhesive ligand presentation on fibroblast spread area and shape was also investigated. Increased spreading was used as a proxy for increased cell contractility and myofibroblast-like activation as previously observed in many in vitro systems (Mcbeath et al., 2004; Balestrini et al., 2012; Guvendiren et al., 2014; Yeh et al., 2017; Charrier et al., 2018). Human lung fibroblasts were seeded atop hydrogels and cultured for three days. Fibroblast spread area and cell shape index, a measure of cell circularity between 0 and 1 where 0 is a line and 1 is a circle (FIGS. 12A-12C), were then quantified. For the RGD-presenting hydrogels, cells showed greater spreading (2590±670 μm²) on stiff elastic groups compared to smaller morphologies on soft (1210±650 μm²) and stiff (1110±510 μm²) viscoelastic groups, similar to results observed previously (Hui et al., 2019). The promotion of α5β1 engagement largely blunted the stiffness-dependent spreading response with fibroblasts showing reduced spreading and more rounded morphologies across all hydrogel groups regardless of stiffness or viscoelasticity (average spread area on Fn9*10 hydrogels: 780±490 μm²), similar to previous findings with alveolar epithelial cell spreading (Brown et al., 2011; Markowski et al., 2012). Hydrogels supporting preferential αvβ3 integrin engagement promoted similar levels of spreading to RGD-modified substrates, although increased spreading was observed even on soft elastic substrates (2060±640 μm²). However, cells displayed decreased spreading and remained rounded on viscoelastic hydrogels regardless of stiffness. Additionally, nanoindentation was used to measure apical fibroblast stiffness on the different hydrogel formulations and found that fibroblasts were significantly stiffer on stiff elastic hydrogels where they preferentially engaged αvβ3 (RGD, Fn4G groups), but not on Fn9*10-modified hydrogels (FIG. 13).

Similar to previous experimental and theoretical results, reduced cell spreading was observed on stiffer viscoelastic substrates compared to their elastic counterparts (Chaudhuri et al., 2015; Gong et al., 2018). However, in contrast to these findings, decreased spreading on softer viscoelastic substrates was also observed. This can likely be attributed to differences in the viscoelastic hydrogel design; ionically-crosslinked viscoelastic hydrogels enable plastic deformation and adhesive ligand clustering to support increased cell spreading at lower stiffnesses (Chaudhuri et al., 2015). In contrast, the presently disclosed viscoelastic hydrogel contains both covalent and supramolecular crosslinks and does not undergo plastic deformation (see stress relaxation and recovery tests in FIG. 10).

### Example 4

### Preferential αvβ3 Integrin Engagement Promotes Actin Stress Fiber Organization and Larger Focal Adhesion Formation

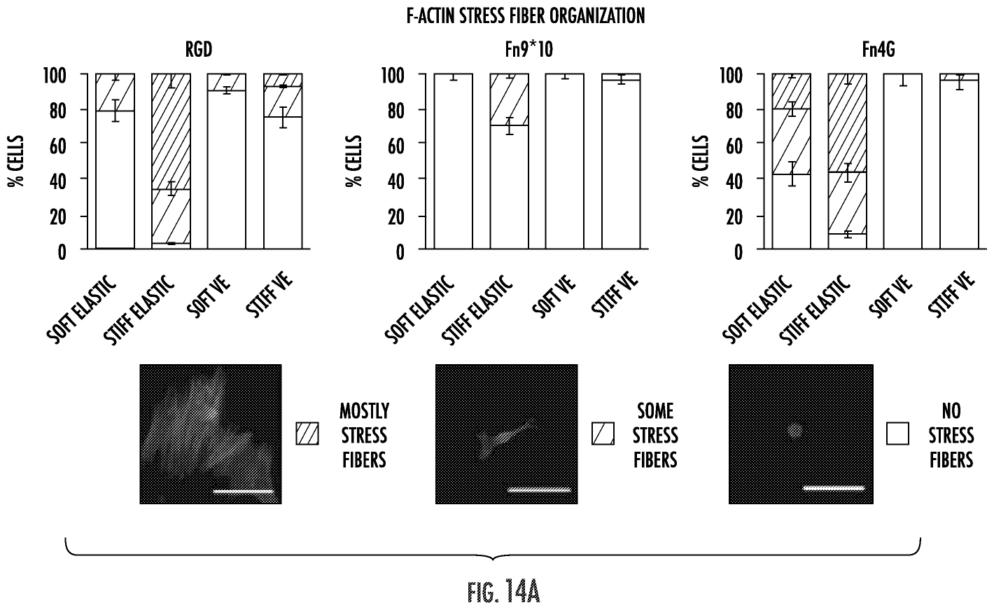

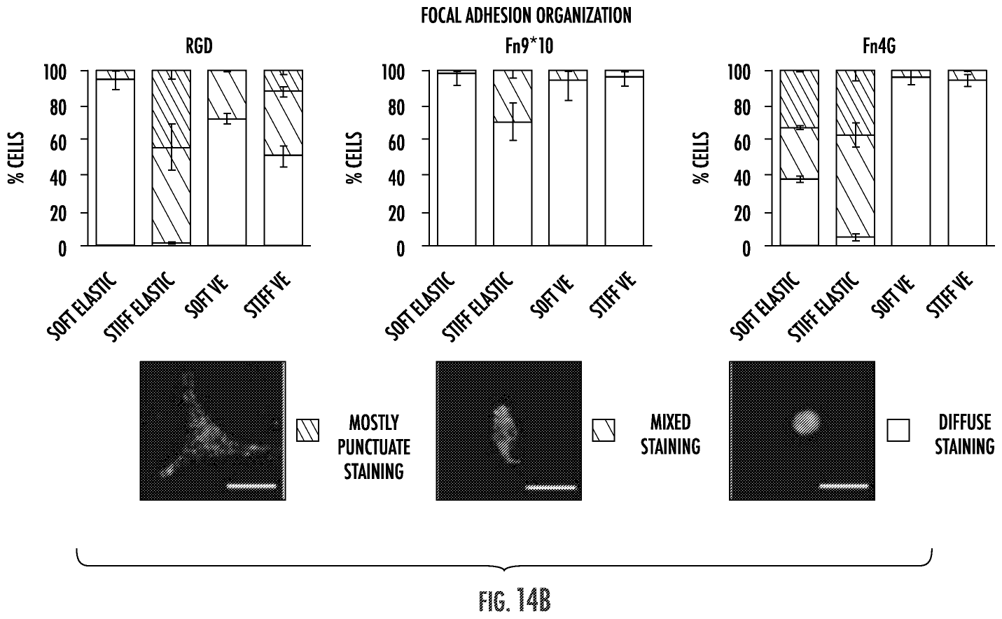

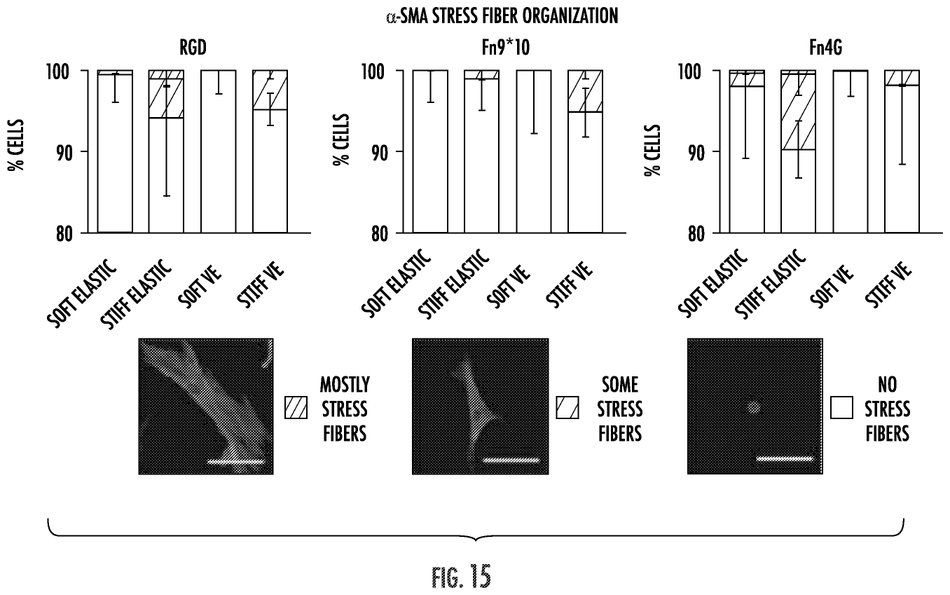

The differences in fibroblast spreading observed as a function of stiffness, viscoelasticity, and adhesive ligand motivated us to more completely understand potential differences in cytoskeletal organization, particularly actin stress fiber formation and focal adhesion maturation. First, the level of actin stress fiber organization as well as the organization of paxillin, a prominent focal adhesion (FA) adaptor protein that has been implicated in regulating cytoskeletal organization (Turner, 2000a; Turner, 2000b; Sero et al., 2012; López-Colomé et al., 2017) was qualitatively evaluated in fibroblasts seeded on hydrogels (FIGS. 14A and 14B). It was found that F-actin organization was strongly correlated to spread area, with significantly more fibroblasts on both stiff elastic RGD and stiff elastic Fn4G hydrogels engaging primarily αvβ3 displaying organized stress fibers. In contrast, fibroblasts on Fn9*10 hydrogels showed few organized stress fibers, even on stiff elastic hydrogels mimicking fibrotic tissue. Notably, F-actin stress fiber organization was absent in the vast majority of fibroblasts cultured on soft or stiff viscoelastic hydrogels regardless of adhesive ligand functionalization. Qualitative analysis of α-SMA, a later marker of the myofibroblast phenotype (Waisberg et al., 2012; Caliari et al., 2016c; Shinde et al., 2017; Hui et al., 2019), showed relatively low levels of stress fiber organization across all hydrogel groups (FIG. 15). This was expected due to the shorter culture time used in this study. Nevertheless, significantly more fibroblasts displaying organized α-SMA stress fibers were observed on stiff elastic RGD substrates.

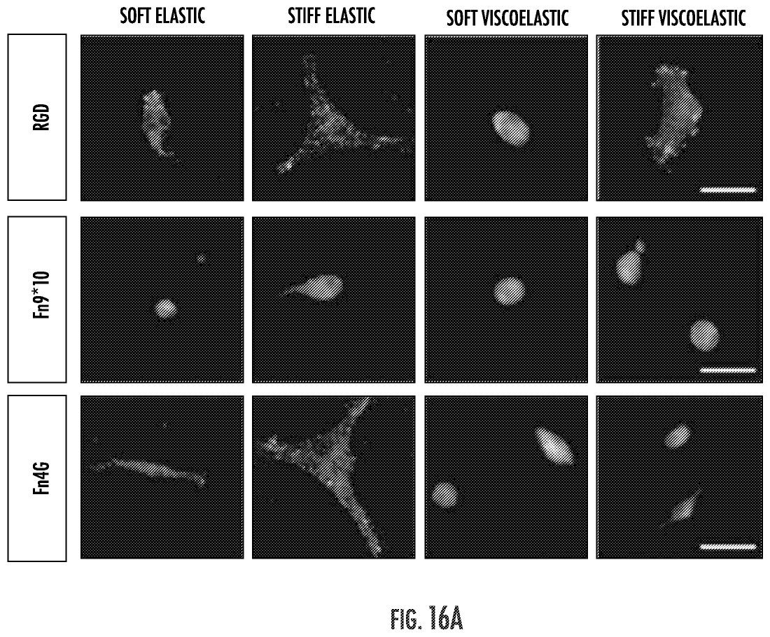

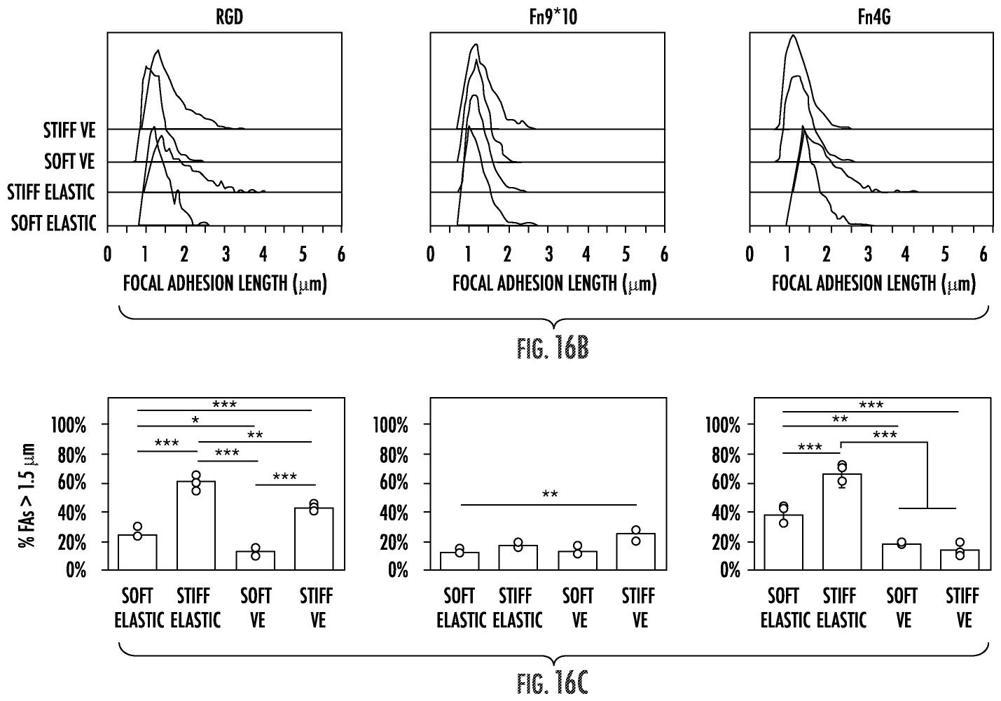

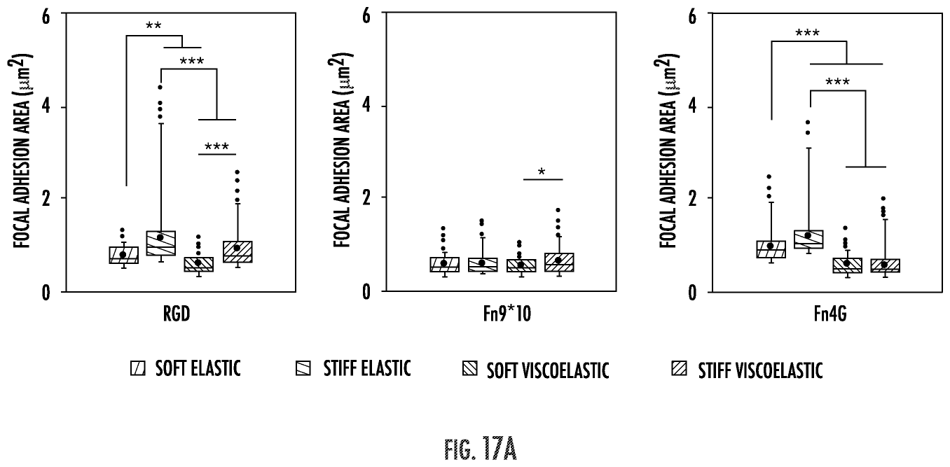

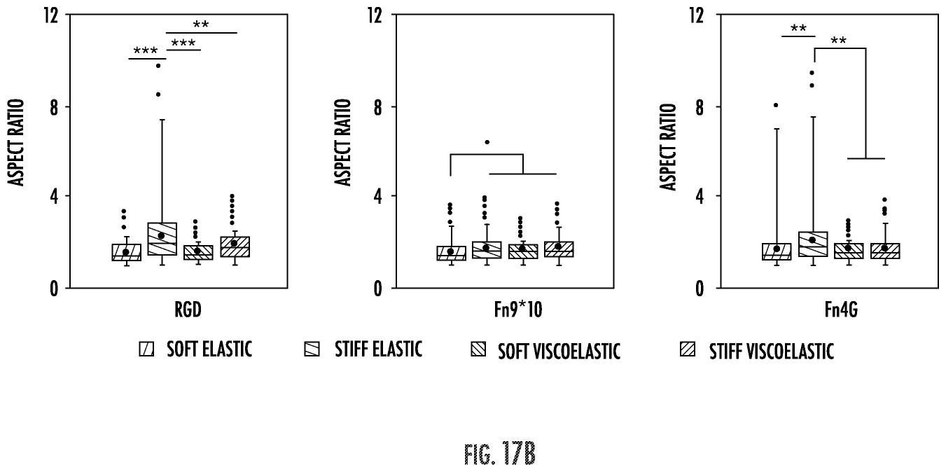

On RGD-containing hydrogels punctate focal adhesion organization, as measured by paxillin staining, was observed near the periphery of the majority of cells on stiff elastic substrates (FIGS. 14B and 16A-16C). In contrast, fibroblasts on soft viscoelastic substrates, more reminiscent of normal healthy soft tissue, contained little to no punctate localization of paxillin, which can be attributed to the increase in viscous character (loss modulus) preventing spreading and the formation of larger FAs. Fibroblasts on soft elastic and stiff viscoelastic substrates displayed a mix of punctate paxillin staining and diffuse staining. Cells on Fn9*10 hydrogels, which typically remained rounded regardless of stiffness or viscoelasticity, showed mainly diffuse paxillin staining. Fibroblasts on Fn4G αvβ3-engaging elastic hydrogels also led to a mix of punctate paxillin structures and diffuse staining, similar to those seen with RGD groups. Again, viscoelasticity played a role in suppressing the formation of larger focal adhesions. These findings were also observed quantitatively with fibroblasts on αvβ3-engaging hydrogels (RGD, Fn4G) displaying increased focal adhesion area (FIGS. 17A and 17B). However, some large, mature FAs were observed for fibroblasts seeded on soft elastic Fn4G hydrogels. Together, these results suggest that preferential αvβ3 binding may facilitate focal adhesion maturation and subsequent actin stress fiber organization and spreading even on soft hydrogels that are more linearly elastic, perhaps mimicking the soft but less viscoelastic mechanical environment observed in active fibroblastic foci in progressive pulmonary fibrosis (Fiore et al., 2018).

### Discussion of the Examples

Described herein is the successful design and implementation of a modular hydrogel platform enabling independent control of covalent crosslinking, incorporation of supramolecular guest-host interactions, and functionalization with cell adhesive groups differentially engaging integrin heterodimers. Hydrogels with stiffnesses approximating normal and fibrotic lung tissue were synthesized in both elastic and viscoelastic forms presenting either RGD or Fn fragments promoting preferential α5β1 or αvβ3 binding. It has been shown that fibroblasts seeded on hydrogels preferentially engaging αvβ3 (RGD, Fn4G) generally showed increased spreading, actin stress fiber formation, and focal adhesion size on stiffer elastic hydrogels, but viscoelasticity played a role in suppressing spreading and focal adhesion maturation regardless of adhesive ligand presentation. In particular, fibrosis-associated αv engagement on Fn4G-modified hydrogels promoted increased spread area and focal adhesion size, even on softer elastic materials. Together, these results highlight the importance of understanding the combinatorial role that viscoelastic and adhesive cues play in regulating fibroblast mechanobiology.

### REFERENCES

All references listed below, as well as all references cited in the instant disclosure, including but not limited to all patents, patent applications and publications thereof, scientific journal articles, and database entries (including all annotations available therein) are incorporated herein by reference in their entireties to the extent that they supplement, explain, provide a background for, or teach methodology, techniques, and/or compositions employed herein.
Aida et al. (2012) Functional supramolecular polymers. Science 335:813-817.
Arora et al. (1999) The Compliance of Collagen Gels Regulates Transforming Growth Factor-β Induction of α-Smooth Muscle Actin in Fibroblasts. Am J Pathol 154:871-882.
Asano et al. (2017) Matrix stiffness regulates migration of human lung fibroblasts. Physiol Rep 5:1-11.
Baker & Chen (2012) Deconstructing the third dimension—how 3D culture microenvironments alter cellular cues. J Cell Sci 125:3015-3024.
Balestrini et al. (2012) The mechanical memory of lung myofibroblasts. Integr Biol 4:410-421.
Berginski & Gomez (2013) The Focal Adhesion Analysis Server: a web tool for analyzing focal adhesion dynamics. F1000Research 2:68.
Booth et al. (2012) Acellular normal and fibrotic human lung matrices as a culture system for in vitro investigation. Am J Respir Crit Care Med 186:866-876.
Brown et al. (2011) Guiding epithelial cell phenotypes with engineered integrin-specific recombinant fibronectin fragments. Tissue Eng.—Part A 17:139-150.
Burdick & Prestwich (2011) Hyaluronic Acid Hydrogels for Biomedical Applications. Adv Mater 23:H41-H56.
Caliari & Burdick (2016) A practical guide to hydrogels for cell culture. Nat. Methods 13:405-414.
Caliari et al. (2016a) Dimensionality and spreading influence MSC YAP/TAZ signaling in hydrogel environments. Biomaterials 103:314-323.
Caliari et al. (2016b) Gradually softening hydrogels for modeling hepatic stellate cell behavior during fibrosis regression. Integr Biol 8:720-728.
Caliari et al. (2016c) Stiffening hydrogels for investigating the dynamics of hepatic stellate cell mechanotransduction during myofibroblast activation. Sci Rep 6:1-10.
Cameron et al. (2011) The influence of substrate creep on mesenchymal stem cell behaviour and phenotype. Biomaterials 32:5979-5993.
Cao et al. (2017) Detection of an Integrin-Binding Mechanoswitch within Fibronectin during Tissue Formation and Fibrosis. ACS Nano 11:7110-7117.
Charrier et al. (2018) Control of cell morphology and differentiation by substrates with independently tunable elasticity and viscous dissipation. Nat Commun 1-13.
Chaudhuri et al. (2015) Substrate stress relaxation regulates cell spreading. Nat Commun 6:6365.
Chaudhuri et al. (2016) Hydrogels with tunable stress relaxation regulate stem cell fate and activity. Nat Mater 15:326-334.
Chaudhuri et al. (2020) Effects of extracellular matrix viscoelasticity on cellular behaviour. Nature 584:535-546.
Conroy et al. (2016) αv integrins: key regulators of tissue fibrosis. Cell Tissue Res 365:511-519.
Deng et al. (2015) Platelet-Derived Growth Factor and Transforming Growth Factor β1 Regulate ARDS-Associated Lung Fibrosis Through Distinct Signaling Pathways. Cell Physiol. Biochem 36:937-946.
Dicker et al. (2014) Hyaluronan: A simple polysaccharide with diverse biological functions. Acta Biomater 10:1558-1570.
Driscoll et al. (2020) Actin flow-dependent and -independent force transmission through integrins. Proc Natl Acad Sci 117:32413-32422.
Duscher et al. (2014) Mechanotransduction and fibrosis. J Biomech 47:1997-2005.
Fernandez & Eickelberg (2012) The Impact of TGF-b on Lung Fibrosis From Targeting to Biomarkers. Proc Am Thorac Soc 9:111-116.
Fiore et al. (2018) Integrin avb3 drives fibroblast contraction and strain stiffening of soft provisional extracellular matrix during progressive fibrosis. JCI Insight 3:1-35.
Gardel et al. (2010) Mechanical Integration of Actin and Adhesion Dynamics in Cell Migration. Annu Rev Cell Dev Biol 26:315-333.
Gong et al. (2018) Matching material and cellular timescales maximizes cell spreading on viscoelastic substrates. Proc Natl Acad Sci 115:E2686-E2695.
Gramlich et al. (2013) Synthesis and orthogonal photopatterning of hyaluronic acid hydrogels with thiol-norbornene chemistry. Biomaterials 34:9803-9811.
Guvendiren et al. (2014) Hydrogels with differential and patterned mechanics to study stiffness-mediated myofibroblastic differentiation of hepatic stellate cells. J Mech Behav Biomed Mater 38:198-208.
Harjanto & Zaman (2010) Matrix mechanics and receptor-ligand interactions in cell adhesion. Org Biomol Chem 8:299-304.
Hautanen et al. (1989) Effects of Modifications of the RGD Sequence and Its Context on Recognition by the Fibronectin Receptor. J Biol Chem 264:1437-1442.
Henderson et al. (2013) Targeting of αv integrin identifies a core molecular pathway that regulates fibrosis in several organs. Nat Med 19:1617-1624.
Henderson et al. (2020) Fibrosis: from mechanisms to medicines. Nature 587:555-566.
Henderson & Sheppard (2013) Integrin-mediated regulation of TGFβ in fibrosis. Biochim Biophys Acta 1832:891-896.
Hilster et al. (2020) Human lung extracellular matrix hydrogels resemble the stiffness and viscoelasticity of native lung tissue. Am J Physiol Lung Cell Mol Physiol 318(4):L698-L704.
Hinz & Gabbiani (2003) Mechanisms of force generation and transmission by myofibroblasts. Curr Opin Biotechnol 14:538-546.
Hui et al. (2019) Spatiotemporal control of viscoelasticity in phototunable hyaluronic acid hydrogels. Biomacromolecules 20:4126-4134.
Humphrey et al. (2014) Mechanotransduction and extracellular matrix homeostasis. Nat Rev Mol Cell Biol 15:802-812.
Ingber (2006) Cellular mechanotransduction: putting all the pieces together again. FASEB J 20:811-827.
Jansen et al. (2017) Mechanotransduction at the cell-matrix interface. Semin Cell Dev Biol 71:75-83.
Kechagia et al. (2019) Integrins as biomechanical sensors of the microenvironment. Nat Rev Mol Cell Biol 20:457-473.
Kishi et al. (2016) Myocardin-related transcription factor A (MRTF-A) activity-dependent cell adhesion is correlated to focal adhesion kinase (FAK) activity. Oncotarget 7:72113-72130.
Kloxin et al. (2010) In situ elasticity modulation with dynamic substrates to direct cell phenotype. Biomaterials 31:1-8.
Kwon et al. (2019) Influence of hyaluronic acid modification on CD44 binding towards the design of hydrogel biomaterials. Biomaterials 222:119451.
Leiphart et al. (2019) Mechanosensing at cellular interfaces. Langmuir 35:7509-7519.
Li et al. (2017) Hydrogels with precisely controlled integrin activation dictate vascular patterning and permeability. Nat Mater 16:953-961.
Liu et al. (2010) Feedback amplification of fibrosis through matrix stiffening and COX-2 suppression. J Cell Biol 190:693-706.
López-Colomé et al. (2017) Paxillin: A crossroad in pathological cell migration. J Hematol Oncol 10:1-15.
Markowski et al. (2012) Directing epithelial to mesenchymal transition through engineered microenvironments displaying orthogonal adhesive and mechanical cues. J Biomed Mater Res—Part A 100A:2119-2127.
Marozas et al. (2019) Adaptable boronate ester hydrogels with tunable viscoelastic spectra to probe timescale dependent mechanotransduction. Biomaterials 223:119430.
Martinez et al. (2017) Idiopathic pulmonary fibrosis. Nat Rev Dis Primer 3:17074.
Martino et al. (2009) Controlling integrin specificity and stem cell differentiation in 2D and 3D environments through regulation of fibronectin domain stability. Biomaterials 30:1089-1097.
Mcbeath et al. (2004) Cell Shape, Cytoskeletal Tension, and RhoA Regulate Stem Cell Lineage Commitment. Dev Cell 6:483-495.
McKinnon et al. (2014) Biophysically defined and cytocompatible covalently adaptable networks as viscoelastic 3D cell culture systems. Adv Mater 26:865-872.
Mohammadi et al. (2015) Inelastic behaviour of collagen networks in cell-matrix interactions and mechanosensation. J R Soc Interface 12:(102)20141074.
Nam et al. (2016) Strain-enhanced stress relaxation impacts nonlinear elasticity in collagen gels. Proc Natl Acad Sci113:1-6.
Olsen et al. (2011) Hepatic stellate cells require a stiff environment for myofibroblastic differentiation. Am J Physiol Gastroinstest Liver Physiol 301:110-118.
Panciera et al. (2017) Mechanobiology of YAP and TAZ in physiology and disease. Nat. Rev. Mol. Cell Biol. 18:758-770.
Paszek et al. (2009) Integrin Clustering Is Driven by Mechanical Resistance from the Glycocalyx and the Substrate. PLoS Comput Biol 5:e1000604.
Perepelyuk et al. (2016) Normal and fibrotic rat livers demonstrate shear strain softening and compression stiffening: A model for soft tissue mechanics. PLoS ONE 11:1-18.
Islam et al. (2020) Micromechanical poroelastic and viscoelastic properties of ex-vivo soft tissues. J Biomech 113:110090.
Roca-Cusachs et al. (2012) Finding the weakest link—exploring integrin-mediated mechanical molecular pathways. J Cell Sci 125:3025-3038.
Rodell et al. (2015) Shear-Thinning Supramolecular Hydrogels with Secondary Autonomous Covalent Crosslinking to Modulate Viscoelastic Properties In Vivo. Adv Funct Mater 25:636-644.
Rodell et al. (2016) Injectable and Cytocompatible Tough Double-Network Hydrogels through Tandem Supramolecular and Covalent Crosslinking. Adv Mater 28:8419-8424.
Rodell et al. (2013) Rational Design of Network Properties in Guest-Host Assembled and Shear-Thinning Hyaluronic Acid Hydrogels. Biomacromolecules 14:4125-4134.
Ruoslahti (1996) RGD and Other Recognition Sequences for Integrins. Annu Rev Cell Dev Biol 12:697-715.
Rustad et al. (2013) The role of focal adhesion complexes in fibroblast mechanotransduction during scar formation. Differentiation 86:87-91.
Scaffidi et al. (2001) Regulation of human lung fibroblast phenotype and function by vitronectin and vitronectin integrins. J Cell Sci 114:3507-3516.
Schanté et al. (2011) Chemical modifications of hyaluronic acid for the synthesis of derivatives for a broad range of biomedical applications. Carbohydr Polym 85:469-489.
Seong et al. (2013) Mechanotransduction at focal adhesions: from physiology to cancer development J Cell Mol Med 17:597-604.
Sero et al. (2012) Paxillin controls directional cell motility in response to physical cues. Cell Adhes Migr 6:502-508.
Shinde et al. (2017) The role of α-smooth muscle actin in fibroblast-mediated matrix contraction and remodeling. Biochim Biophys Acta 1863:298-309.
Sun et al. (2016) Integrin-mediated mechanotransduction. J Cell Bio 215:445-456.
Tang et al. (2018) Adaptable Fast Relaxing Boronate-Based Hydrogels for Probing Cell-Matrix Interactions. Adv Sci 5(9):1800638.
Travers et al. (2016) The Fibroblast Awakens. Circ Res 118(6):1021-1040.
Turner (2000a) Paxillin and focal adhesion signaling. Nat Cell Biol 2:231-236.
Turner (2000b) Paxillin interactions. J Cell Sci 113:4139-4140.
van de Manakker et al. (2010) Supramolecular hydrogels formed by β-cyclodextrin self-association and host-guest inclusion complexes. Soft Matter 6:187-194.
Waisberg et al. (2012) Increased fibroblast telomerase expression precedes myofibroblast a-smooth muscle actin expression in idiopathic pulmonary fibrosis. Clinics 67:1039-1046.
Wells (2008) The role of matrix stiffness in regulating cell behavior. Hepatology 47:1394-1400.
Wells (2014) Tissue Mechanics and Fibrosis. Biochim Biophys Acta 1832:884-890.
Wen et al. (2014) Interplay of matrix stiffness and protein tethering in stem cell differentiation. Nat Mater 13:979-987.
Wynn (2008) Cellular and molecular mechanisms of fibrosis. J. Pathol 214:199-210.
Wynn & Ramalingam (2012) Mechanisms of fibrosis: Therapeutic translation for fibrotic disease. Nat Med 18:1028-1040.
Yeh et al. (2017) Mechanically dynamic PDMS substrates to investigate changing cell environments. Biomaterials 145:23-32.
Zhao et al. (2010) Stress-relaxation behavior in gels with ionic and covalent crosslinks. J Appl Phys 107:1-5.
Zhu et al. (2020) Spatial mapping of tissue properties in vivo reveals a 3D stiffness gradient in the mouse limb bud. Proc Natl Acad Sci 117:4781.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A phototunable hydrogel comprising:
a norbornene-functionalized hyaluronic acid (HA) backbone and optionally one or more additional functional moieties attached thereto, wherein the one or more additional functional moieties are selected from the group consisting of β-cyclodextrin and adamantane, or any combination thereof; and
(ii) one or more peptides and/or polypeptide fragments, wherein the one or more peptides and/or polypeptide fragments are selected from the group consisting of an RGD peptide and a fibronectin polypeptide fragment, optionally wherein the fibronectin polypeptide fragment is selected from the group consisting of an α5β1 peptide and an αvβ3 peptide, or any combination thereof, and further optionally wherein the fibronectin polypeptide fragment is thiolated.

2. The phototunable hydrogel of claim 1, wherein at least two norbornene moieties are crosslinked to each other, optionally with a dithiol crosslinker.

3. The phototunable hydrogel of claim 2, wherein the dithiol crosslinker comprises a covalent crosslink that results from light-mediated thiol-ene addition.

4. The phototunable hydrogel of claim 1, wherein the norbornene-functionalized HA backbone lacks β-cyclodextrin and adamantane and the phototunable hydrogel is an elastic phototunable hydrogel.

5. The phototunable hydrogel of claim 1, wherein the norbornene-functionalized HA backbone comprises one or more β-cyclodextrin and/or adamantane moieties, optionally thiolated adamantane moieties, and the phototunable hydrogel is a viscoelastic phototunable hydrogel.

6. The phototunable hydrogel of claim 5, wherein the phototunable hydrogel comprises a plurality of β-cyclodextrin moieties and a plurality of thiolated adamantane moieties, and at least a subset of the β-cyclodextrin moieties and the thiolated adamantane moieties form supramolecular guest-host interactions in order to confer viscosity to the phototunable hydrogel.

7. The phototunable hydrogel of claim 1, wherein the phototunable hydrogel has a Young's modulus of less than about 5 kPa, optionally of about 0.5-1.0 kPa.

8. The phototunable hydrogel of claim 1, wherein the phototunable hydrogel has a Young's modulus of about at least about 5 kPa, optionally of at least about 10 kPa, further optionally of at least about 15 kPa.

9. The phototunable hydrogel of claim 1, wherein the RGD peptide comprises, consists essentially of, or consists of the amino acid sequence GCGYGRGDSPG (SEQ ID NO: 3).

10. A method for treating a wound or injury in a subject in need thereof, the method comprising:
(a) administering to a site of a wound or injury an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel at the site of the wound or injury,
wherein the presence of the cured phototunable hydrogel at the site of the wound or injury enhances recovery of the wound or injury to thereby treat the wound or injury in the subject.

11. The method of claim 10, wherein the wound is a superficial wound or injury and the composition is administered topically and then exposed to the light source.

12. The method of claim 10, wherein the wound is an internal wound or injury and the composition comprising the phototunable hydrogel of claim 1 is administered by injection and then exposed to the light source at the site of the internal wound or injury.

13. The method of claim 12, wherein the internal wound or injury is a muscle injury.

14. The method of claim 12, further comprising inserting a physical barrier around the site of the internal wound or injury prior to administering the composition, wherein the physical barrier retains the administered composition at the site of the internal wound or injury for at least a time before the phototunable hydrogel is cured at the site of the wound or injury.

15. The method of claim 10, wherein the light source provides a light wavelength of about 365-505 nm, a power density of about 2-15 mW/cm², or both.

16. The method of claim 10, wherein the exposing step is for a duration of about 2-10 minutes.

17. The method of claim 10, wherein the cured phototunable hydrogel inhibits myofibroblast formation at the site of the wound or injury.

18. A method for inhibiting formation of scar tissue at a wound site of a subject in need thereof, the method comprising:
(a) administering to the wound site an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel at the wound site,
wherein the presence of the cured phototunable hydrogel at the wound site inhibits formation of scar tissue at the wound site.

19. A method for inhibiting fibrosis in a subject in need thereof, the method comprising:
(a) administering to a site expected to undergo fibrosis in the subject an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel at the site expected to undergo fibrosis,
wherein the presence of the cured phototunable hydrogel at the site expected to undergo fibrosis inhibits fibrosis in the subject.

20. A method for inhibiting lung fibrosis and/or scarring in a subject in need thereof, the method comprising:
(a) administering to a site in a lung of the subject an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel at the site in the lung,
whereby presence of the cured phototunable hydrogel at the site in the lung inhibits formation of lung fibrosis and/or scarring in the subject.

21. The method of claim 10, wherein the subject is a mammal, optionally a mouse or a human.

22. The method of claim 10, wherein the method further comprises providing a photomask to at least a part of the site to provide spatiotemporal control of where covalent and/or supramolecular crosslinks occur in the hydrogel at the site.

23. The method of claim 10, wherein the administering step is repeated one or more times.

24. A method for inhibiting formation of a myofibroblast from a fibroblast, the method comprising:
(a) contacting the fibroblast with an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel,
whereby the presence of the cured phototunable hydrogel inhibits formation of a myofibroblast from the fibroblast.

25. A method for inhibiting expression of α-smooth muscle actin (α-SMA) and/or type I collagen in a fibroblast, the method comprising:
(a) contacting the fibroblast with an effective amount of a composition comprising a phototunable hydrogel of claim 1; and
(b) exposing the composition to a photoinitiator, optionally lithium acylphosphinate, and a light source in an amount and for a time sufficient to cure the phototunable hydrogel,
whereby the presence of the cured phototunable hydrogel inhibits expression of α-smooth muscle actin (α-SMA) and/or type I collagen in the fibroblast.

26. The method of claim 24, wherein the fibroblast is present within a subject, optionally a human.

27. The method of claim 10, wherein the phototunable hydrogel is a soft viscoelastic hydrogel functionalized with one or more Fn9*10 fibronectin fragments.

28. The method of claim 10, wherein the phototunable hydrogel comprises a plurality of norbornene moieties, at least two of which are crosslinked to each other, optionally with a dithiol crosslinker, further optionally wherein the dithiol crosslinker comprises a covalent crosslink that results from light-mediated thiol-ene addition.

29. The method of claim 28, wherein at least one dithiol crosslinker comprises an enzymatically-degradable peptide to thereby allow the phototunable hydrogel to degrade over time.


SEQUENCE LISTING (4 sequences)

SEQ ID NO: 1 - protein, 5 aa, Artificial Sequence
  Artificially synthesized peptide
KKKCG

SEQ ID NO: 2 - protein, 5 aa, Homo sapiens
PHSRN

SEQ ID NO: 3 - protein, 11 aa, Artificial Sequence
  Artificially synthesized peptide
GCGYGRGDSPG

SEQ ID NO: 4 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE